SEQUENCE LISTING (6 sequences)

SEQ ID NO: 1 - DNA, 2076 bp, Homo sapiens
  CDS (1)..(2073)
tgcaccacccctggctgcgtgatagcagctgccaggatcctccagaacatggacccgaccacggaaccgtgtgacgacttctaccagtttgcatgcggaggctggctgcggcgccacgtgatccctgagaccaactcaagatacagcatctttgacgtcctccgcgacgagctggaggtcatcctcaaagcggtgctggagaattcgactgccaaggaccggccggctgtggagaaggccaggacgctgtaccgctcctgcatgaaccagagtgtgatagagaagcgaggctctcagcccctgctggacatcttggaggtggtgggaggctggccggtggcgatggacaggtggaacgagaccgtaggactcgagtgggagctggagcggcagctggcgctgatgaactcacagttcaacaggcgcgtcctcatcgacctcttcatctggaacgacgaccagaactccagccggcacatcatctacatagaccagcccaccttgggcatgccctcccgagagtactacttcaacggcggcagcaaccggaaggtgcgggaagcctacctgcagttcatggtgtcagtggccacgttgctgcgggaggatgcaaacctgcccagggacagctgcctggtgcaggaggacatgatgcaggtgctggagctggagacacagctggccaaggccacggtaccccaggaggagagacacgacgtcatcgccttgtaccaccggatgggactggaggagctgcaaagccagtttggcctgaagggatttaactggactctgttcatacaaactgtgctatcctctgtcaaaatcaagctgctgccagatgaggaagtggtggtctatggcatcccctacctgcagaaccttgaaaacatcatcgacacctactcagccaggaccatacagaactacctggtctggcgcctggtgctggaccgcattggtagcctaagccagagattcaaggacacacgagtgaactaccgcaaggcgctgtttggcacaatggtggaggaggtgcgctggcgtgaatgtgtgggctacgtcaacagcaacatggagaacgccgtgggctccctctacgtcagggaggcgttccctggagacagcaagagcatggtcagagaactcattgacaaggtgcggacagtgtttgtggagacgctggacgagctgggctggatggacgaggagtccaagaagaaggcgcaggagaaggccatgagcatccgggagcagatcgggcaccctgactacatcctggaggagatgaacaggcgcctggacgaggagtactccaatctgaacttctcagaggacctgtactttgagaacagtctgcagaacctcaaggtgggcgcccagcggagcctcaggaagcttcgggaaaaggtggacccaaatctctggatcatcggggcggcggtggtcaatgcgttctactccccaaaccgaaaccagattgtattccctgccgggatcctccagccccccttcttcagcaaggagcagccacaggccttgaactttggaggcattgggatggtgatcgggcacgagatcacgcacggctttgacgacaatggccggaacttcgacaagaatggcaacatgatggattggtggagtaacttctccacccagcacttccgggagcagtcagagtgcatgatctaccagtacggcaactactcctgggacctggcagacgaacagaacgtgaacggattcaacacccttggggaaaacattgctgacaacggaggggtgcggcaagcctataaggcctacctcaagtggatggcagagggtggcaaggaccagcagctgcccggcctggatctcacccatgagcagctcttcttcatcaactacgcccaggtgtggtgcgggtcctaccggcccgagttcgccatccaatccatcaagacagacgtccacagtcccctgaagtacagggtactggggtcgctgcagaacctggccgccttcgcagacacgttccactgtgcccggggcacccccatgcaccccaaggagcgatgccgcgtgtggtag

SEQ ID NO: 2 - protein, 691 aa, Homo sapiens
CTTPGCVIAAARILQNMDPTTEPCDDFYQFACGGWLRRHVIPETNSRYSIFDVLRDELEVILKAVLENSTAKDRPAVEKARTLYRSCMNQSVIEKRGSQPLLDILEVVGGWPVAMDRWNETVGLEWELERQLALMNSQFNRRVLIDLFIWNDDQNSSRHIIYIDQPTLGMPSREYYFNGGSNRKVREAYLQFMVSVATLLREDANLPRDSCLVQEDMMQVLELETQLAKATVPQEERHDVIALYHRMGLEELQSQFGLKGFNWTLFIQTVLSSVKIKLLPDEEVVVYGIPYLQNLENIIDTYSARTIQNYLVWRLVLDRIGSLSQRFKDTRVNYRKALFGTMVEEVRWRECVGYVNSNMENAVGSLYVREAFPGDSKSMVRELIDKVRTVFVETLDELGWMDEESKKKAQEKAMSIREQIGHPDYILEEMNRRLDEEYSNLNFSEDLYFENSLQNLKVGAQRSLRKLREKVDPNLWIIGAAVVNAFYSPNRNQIVFPAGILQPPFFSKEQPQALNFGGIGMVIGHEITHGFDDNGRNFDKNGNMMDWWSNFSTQHFREQSECMIYQYGNYSWDLADEQNVNGFNTLGENIADNGGVRQAYKAYLKWMAEGGKDQQLPGLDLTHEQLFFINYAQVWCGSYRPEFAIQSIKTDVHSPLKYRVLGSLQNLAAFADTFHCARGTPMHPKERCRVW

SEQ ID NO: 3 - DNA, 2340 bp, Homo Sapiens
  CDS (1)..(2337)
atggggaagtccgaaggccccgtggggatggtggagagcgctggccgtgcagggcagaagcgcccggggttcctggagggggggctgctgctgctgctgctgctggtgaccgctgccctggtggccttgggtgtcctctacgccgaccgcagagggaagcagctgccacgccttgctagccggctgtgcttcttacaggaggagaggacctttgtaaaacgaaaaccccgagggatcccagaggcccaagaggtgagcgaggtctgcaccacccctggctgcgtgatagcagctgccaggatcctccagaacatggacccgaccacggaaccgtgtgacgacttctaccagtttgcatgcggaggctggctgcggcgccacgtgatccctgagaccaactcaagatacagcatctttgacgtcctccgcgacgagctggaggtcatcctcaaagcggtgctggagaattcgactgccaaggaccggccggctgtggagaaggccaggacgctgtaccgctcctgcatgaaccagagtgtgatagagaagcgaggctctcagcccctgctggacatcttggaggtggtgggaggctggccggtggcgatggacaggtggaacgagaccgtaggactcgagtgggagctggagcggcagctggcgctgatgaactcacagttcaacaggcgcgtcctcatcgacctcttcatctggaacgacgaccagaactccagccggcacatcatctacatagaccagcccaccttgggcatgccctcccgagagtactacttcaacggcggcagcaaccggaaggtgcgggaagcctacctgcagttcatggtgtcagtggccacgttgctgcgggaggatgcaaacctgcccagggacagctgcctggtgcaggaggacatgatgcaggtgctggagctggagacacagctggccaaggccacggtaccccaggaggagagacacgacgtcatcgccttgtaccaccggatgggactggaggagctgcaaagccagtttggcctgaagggatttaactggactctgttcatacaaactgtgctatcctctgtcaaaatcaagctgctgccagatgaggaagtggtggtctatggcatcccctacctgcagaaccttgaaaacatcatcgacacctactcagccaggaccatacagaactacctggtctggcgcctggtgctggaccgcattggtagcctaagccagagattcaaggacacacgagtgaactaccgcaaggcgctgtttggcacaatggtggaggaggtgcgctggcgtgaatgtgtgggctacgtcaacagcaacatggagaacgccgtgggctccctctacgtcagggaggcgttccctggagacagcaagagcatggtcagagaactcattgacaaggtgcggacagtgtttgtggagacgctggacgagctgggctggatggacgaggagtccaagaagaaggcgcaggagaaggccatgagcatccgggagcagatcgggcaccctgactacatcctggaggagatgaacaggcgcctggacgaggagtactccaatctgaacttctcagaggacctgtactttgagaacagtctgcagaacctcaaggtgggcgcccagcggagcctcaggaagcttcgggaaaaggtggacccaaatctctggatcatcggggcggcggtggtcaatgcgttctactccccaaaccgaaaccagattgtattccctgccgggatcctccagccccccttcttcagcaaggagcagccacaggccttgaactttggaggcattgggatggtgatcgggcacgagatcacgcacggctttgacgacaatggccggaacttcgacaagaatggcaacatgatggattggtggagtaacttctccacccagcacttccgggagcagtcagagtgcatgatctaccagtacggcaactactcctgggacctggcagacgaacagaacgtgaacggattcaacacccttggggaaaacattgctgacaacggaggggtgcggcaagcctataaggcctacctcaagtggatggcagagggtggcaaggaccagcagctgcccggcctggatctcacccatgagcagctcttcttcatcaactacgcccaggtgtggtgcgggtcctaccggcccgagttcgccatccaatccatcaagacagacgtccacagtcccctgaagtacagggtactggggtcgctgcagaacctggccgccttcgcagacacgttccactgtgcccggggcacccccatgcaccccaaggagcgatgccgcgtgtggtag

SEQ ID NO: 4 - protein, 779 aa, Homo Sapiens
MGKSEGPVGMVESAGRAGQKRPGFLEGGLLLLLLLVTAALVALGVLYADRRGKQLPRLASRLCFLQEERTFVKRKPRGIPEAQEVSEVCTTPGCVIAAARILQNMDPTTEPCDDFYQFACGGWLRRHVIPETNSRYSIFDVLRDELEVILKAVLENSTAKDRPAVEKARTLYRSCMNQSVIEKRGSQPLLDILEVVGGWPVAMDRWNETVGLEWELERQLALMNSQFNRRVLIDLFIWNDDQNSSRHIIYIDQPTLGMPSREYYFNGGSNRKVREAYLQFMVSVATLLREDANLPRDSCLVQEDMMQVLELETQLAKATVPQEERHDVIALYHRMGLEELQSQFGLKGFNWTLFIQTVLSSVKIKLLPDEEVVVYGIPYLQNLENIIDTYSARTIQNYLVWRLVLDRIGSLSQRFKDTRVNYRKALFGTMVEEVRWRECVGYVNSNMENAVGSLYVREAFPGDSKSMVRELIDKVRTVFVETLDELGWMDEESKKKAQEKAMSIREQIGHPDYILEEMNRRLDEEYSNLNFSEDLYFENSLQNLKVGAQRSLRKLREKVDPNLWIIGAAVVNAFYSPNRNQIVFPAGILQPPFFSKEQPQALNFGGIGMVIGHEITHGFDDNGRNFDKNGNMMDWWSNFSTQHFREQSECMIYQYGNYSWDLADEQNVNGFNTLGENIADNGGVRQAYKAYLKWMAEGGKDQQLPGLDLTHEQLFFINYAQVWCGSYRPEFAIQSIKTDVHSPLKYRVLGSLQNLAAFADTFHCARGTPMHPKERCRVW

SEQ ID NO: 5 - DNA, 2262 bp, Homo Sapiens
  CDS (1)..(2259)
atggggaagtccgaaggcccagtggggatggtggagagcgccggccgtgcagggcagaagcgcccggggttcctggagggggggctgctgctgctgctgctgctggtgaccgctgccctggtggccttgggtgtcctctacgccgaccgcagagggatcccagaggcccaagaggtgagcgaggtctgcaccacccctggctgcgtgatagcagctgccaggatcctccagaacatggacccgaccacggaaccgtgtgacgacttctaccagtttgcatgcggaggctggctgcggcgccacgtgatccctgagaccaactcaagatacagcatctttgacgtcctccgcgacgagctggaggtcatcctcaaagcggtgctggagaattcgactgccaaggaccggccggctgtggagaaggccaggacgctgtaccgctcctgcatgaaccagagtgtgatagagaagcgaggctctcagcccctgctggacatcttggaggtggtgggaggctggccggtggcgatggacaggtggaacgagaccgtaggactcgagtgggagctggagcggcagctggcgctgatgaactcacagttcaacaggcgcgtcctcatcgacctcttcatctggaacgacgaccagaactccagccggcacatcatctacatagaccagcccaccttgggcatgccctcccgagagtactacttcaacggcggcagcaaccggaaggtgcgggaagcctacctgcagttcatggtgtcagtggccacgttgctgcgggaggatgcaaacctgcccagggacagctgcctggtgcaggaggacatgatgcaggtgctggagctggagacacagctggccaaggccacggtaccccaggaggagagacacgacgtcatcgccttgtaccaccggatgggactggaggagctgcaaagccagtttggcctgaagggatttaactggactctgttcatacaaactgtgctatcctctgtcaaaatcaagctgctgccagatgaggaagtggtggtctatggcatcccctacctgcagaaccttgaaaacatcatcgacacctactcagccaggaccatacagaactacctggtctggcgcctggtgctggaccgcattggtagcctaagccagagattcaaggacacacgagtgaactaccgcaaggcgctgtttggcacaatggtggaggaggtgcgctggcgtgaatgtgtgggctacgtcaacagcaacatggagaacgccgtgggctccctctacgtcagggaggcgttccctggagacagcaagagcatggtcagagaactcattgacaaggtgcggacagtgtttgtggagacgctggacgagctgggctggatggacgaggagtccaagaagaaggcgcaggagaaggccatgagcatccgggagcagatcgggcaccctgactacatcctggaggagatgaacaggcgcctggacgaggagtactccaatctgaacttctcagaggacctgtactttgagaacagtctgcagaacctcaaggtgggcgcccagcggagcctcaggaagcttcgggaaaaggtggacccaaatctctggatcatcggggcggcggtggtcaatgcgttctactccccaaaccgaaaccagattgtattccctgccgggatcctccagccccccttcttcagcaaggagcagccacaggccttgaactttggaggcattgggatggtgatcgggcacgagatcacgcacggctttgacgacaatggccggaacttcgacaagaatggcaacatgatggattggtggagtaacttctccacccagcacttccgggagcagtcagagtgcatgatctaccagtacggcaactactcctgggacctggcagacgaacagaacgtgaacggattcaacacccttggggaaaacattgctgacaacggaggggtgcggcaagcctataaggcctacctcaagtggatggcagagggtggcaaggaccagcagctgcccggcctggatctcacccatgagcagctcttcttcatcaactacgcccaggtgtggtgcgggtcctaccggcccgagttcgccatccaatccatcaagacagacgtccacagtcccctgaagtacagggtactggggtcgctgcagaacctggccgccttcgcagacacgttccactgtgcccggggcacccccatgcaccccaaggagcgatgccgcgtgtggtag

SEQ ID NO: 6 - protein, 753 aa, Homo Sapiens
MGKSEGPVGMVESAGRAGQKRPGFLEGGLLLLLLLVTAALVALGVLYADRRGIPEAQEVSEVCTTPGCVIAAARILQNMDPTTEPCDDFYQFACGGWLRRHVIPETNSRYSIFDVLRDELEVILKAVLENSTAKDRPAVEKARTLYRSCMNQSVIEKRGSQPLLDILEVVGGWPVAMDRWNETVGLEWELERQLALMNSQFNRRVLIDLFIWNDDQNSSRHIIYIDQPTLGMPSREYYFNGGSNRKVREAYLQFMVSVATLLREDANLPRDSCLVQEDMMQVLELETQLAKATVPQEERHDVIALYHRMGLEELQSQFGLKGFNWTLFIQTVLSSVKIKLLPDEEVVVYGIPYLQNLENIIDTYSARTIQNYLVWRLVLDRIGSLSQRFKDTRVNYRKALFGTMVEEVRWRECVGYVNSNMENAVGSLYVREAFPGDSKSMVRELIDKVRTVFVETLDELGWMDEESKKKAQEKAMSIREQIGHPDYILEEMNRRLDEEYSNLNFSEDLYFENSLQNLKVGAQRSLRKLREKVDPNLWIIGAAVVNAFYSPNRNQIVFPAGILQPPFFSKEQPQALNFGGIGMVIGHEITHGFDDNGRNFDKNGNMMDWWSNFSTQHFREQSECMIYQYGNYSWDLADEQNVNGFNTLGENIADNGGVRQAYKAYLKWMAEGGKDQQLPGLDLTHEQLFFINYAQVWCGSYRPEFAIQSIKTDVHSPLKYRVLGSLQNLAAFADTFHCARGTPMHPKERCRVW

(19) United States
(12) Patent Application Publication
(10) Pub. No.: US 2004/0162345 A1
(43) Pub. Date: Aug. 19, 2004

(54) Compounds with NEP/MP-inhibitory activity and uses thereof

(75) Inventors: Claudia Berger, Backnang (DE); Yvan Fischer, Barsinghausen (DE); Dagmar Hoeltje, Gehrden (DE); Harald Waldeck, Isernhagen HB (DE); Michael Weske, Burgdorf (DE); Dieter Ziegler, Hemmingen (DE)

Correspondence Address: CROWELL & MORING LLP, INTELLECTUAL PROPERTY GROUP, P.O. BOX 14300, WASHINGTON, DC, 20044-4300, US

(73) Assignee: Solvay Pharmaceuticals GmbH, Hanover (DE)

(21) Appl. No.: 10/715,112

(22) Filed: Nov. 18, 2003

Related U.S. Application Data
(63) Continuation of application No. PCT/EP02/05259, filed on May 14, 2002.

(30) Foreign Application Priority Data
May 18, 2001 (EP) EP 01112231.4

(51) Int. Cl. A61K 31/185

(52) U.S. Cl. 514/553

(57) ABSTRACT

Compounds having combined, particularly concurrent, inhibitory activity on neutral endopeptidase (NEP) and on a novel metalloprotease designated IGS5, or of a pharmaceutically acceptable salt or solvate or biolabile ester thereof, and related methods for treating and for the manufacture of a pharmaceutical composition for a mammal, preferably a larger mammal such as a human, suffering from or being susceptible to a disease or condition which can be alleviated or inhibited by combined or concurrent inhibition of NEP and IGS5. Treatments for diseases or conditions where big-ET-1 levels are elevated, or where ET-1 is upregulated, where such diseases or conditions can be alleviated or inhibited by combined or concurrent inhibition of NEP and IGS5.

### CROSS-REFERENCE TO RELATED APPLICATIONS

This application is a continuation of International Patent Application No. PCT/EP02/05259, filed May 14, 2002 designating the United States of America, and published in English as WO 02/94176, the entire disclosure of which is incorporated herein by reference. Priority is claimed based on European patent application No. 01 11 2231.4, filed May 18, 2001, and U.S. patent application No. 60/292,337, filed May 22, 2001.

### FIELD OF THE INVENTION

This invention relates to the new medical use of compounds which act as combined or concurrent inhibitors of neutral endopeptidase (NEP) and of a specific metalloprotease (MP) which recently has been cloned and newly identified as a genuine metalloprotease with broad substrate specificity.

### BACKGROUND OF THE INVENTION

Metalloproteases are polypeptides which form a particular family of structurally and functionally related enzymes, e.g. peptidases, which are of pharmaceutical or pharmacological interest in the context of treatment or prophylaxis or inhibition of various diseases. Several diseases have been identified where metalloproteases play a critical role in the pathology of the disease. For example, a number of zinc metalloproteases or particular families of structurally and functionally related enzymes have been identified and characterized in the state of the art, and it has become apparent that the participation of these enzymes, e.g. zinc metalloproteases, plays a role in a diverse array of biological functions encompassing both normal and disease situations. Zinc metalloproteases are subset of such enzymes whose catalytic functions are critically dependent on the zinc ion at the active site. This group of enzymes, which comprises various families classified on the basis of both sequence and structural information, are for example described to be intimately involved in such processes as e.g. embryonic development, cartilage and bone formation, processing of peptide hormones, reproduction, cardiovascular diseases, arthritis and cancer. Thus, there is particular interest in the pharmaceutical art not only to investigate the key roles of each of the metalloproteases and their potential interrelationship in health and disease, but especially also in designing improved therapeutical concepts for the management of diseases involving said metalloproteases.

On the basis of sequence and structural information around the zinc binding site of the zinc metalloproteases these enzymes may be classified into several families which may be further classified into superfamilies such as the “metzincins” (astacin, serratia, reprolysin, matrixin), the “gluzincins” (thermolysin, neprilysin, angiotensin converting enzyme, aminopeptidase), or the “zincins” comprising the superfamilies of metzincins and gluzincins. Such grouping not only aids in the elucidation of common catalytic and biosynthetic processing mechanisms, but also is invaluable in elucidating the function(s) of newly identified proteins which possess similar zinc binding motifs. Some individual examples of metalloproteases, e.g. zinc enzymes, already identified in the state of the art comprise neprilysin, endothelin converting enzyme, angiotensin converting enzyme, thermolysin, aminopeptidase, astacin, serratia, reprolysin, matrixin, insulinase, carboxypeptidase and DD-carboxypeptidase.

Some more specific features and related known activities of particularly interesting metalloprotease subtypes like neutral endopeptidase (NEP), endothelin converting enzyme (ECE), and angiotensin converting enzyme (ACE) may be summarized as follows.

Angiotensin I Converting Enzyme (ACE; peptidyl dipeptidase A; EC 3.4.15.1) is a member of the angiotensin converting enzyme family of zinc metalloproteases. ACE is primarily expressed at the surface of endothelial, epithelial and neuroepithelial cells (somatic ACE) as an ectoenzyme, meaning that it is anchored to the plasma membrane with the bulk of its mass, including its catalytic sites, facing the extracellular milieu. ACE is found in the plasma membrane of vascular endothelial cells, with high levels found at the vascular endothelial surface of the lung such that the active sites of ACE are posed to metabolize circulating substrates. In addition to the endothelial location of ACE, the enzyme is also expressed in the brush borders of absorptive epithelia of the small intestine and the kidney proximal convoluted tubule. ACE is also found in mononuclear cells, such as monocytes after macrophage differentiation and T-lymphocytes, and in fibroblasts. In vitro autoradiography, employing radiolabelled specific ACE inhibitors, and immunohistochemical studies have mapped the principal locations of ACE in the brain. ACE was found primarily in the choroid plexus, which may be the source of ACE in cerebrospinal fluid, ependyma, subformical organ, basal ganglia (caudate-putamen and globus pallidus), substantia nigra and pituitary. A soluble form of ACE has been detected in many biological fluids such as serum, seminal fluid, amniotic fluid and cerebrospinal fluid. The soluble form of ACE appears to be derived from the membrane-bound form of the enzyme in endothelial cells. A main physiological activity of ACE is that it cleaves the C-terminal dipeptide from angiotensin I to produce the potent vasopressor peptide angiotensin II and inactivates the vasodilatory peptide bradykinin by the sequential removal of two C-terminal dipeptides. As a consequence of the involvement of ACE in the metabolism of these two vasoactive peptides angiotensin II and bradykinin, ACE has become a crucial molecular target in the treatment of hypertension and congestive heart failure. This has led to the development of highly potent and specific ACE inhibitors which have become clinically important and widespread as orally active drugs to control these conditions of hypertension and congestive heart failure. Whilst the metabolism of vasoactive peptides remains the best known physiological function of ACE, the enzyme has been also implicated in a range of other physiological processes unrelated to blood pressure regulation such as immunity, reproduction and neuropeptide metabolism due to the localization of ACE and/or the in vitro cleavage of a range of biologically active peptides.

Neutral Endopeptidase (NEP, neprilysin, EC 3.4.24.11) is a zinc metalloprotease and classified as a member of the neprilysin family. NEP was first isolated from the brush border membranes of rabbit kidney. Later, an NEP-like enzyme was identified in rat brain as being involved in the degradation of the opioid peptides, enkephalins. The cloning of the ectoenzyme NEP and subsequent site-directed mutagenesis experiments have shown that, as well as having a similar specificity to thermolysin, it also has a similar active site organization. NEP also shows a thermolysin-like specificity for cleaving peptides on the N-terminal side of hydrophobic residues. With regard to the general distribution of NEP it has been determined in the brain and spinal cord, and lesion and electron microscopic studies generally support a predominantly neuronal localization of NEP, although the enzyme could be present on oligodendrocytes surrounding the fibers of the striato-pallidal and striato-nigral pathways and on Schwann cells in the peripheral nervous system. NEP does not appear to be concentrated on specific membrane interfaces such as the synapse, but is rather uniformly distributed on the surface of neuronal perikarya and dendrites. In the periphery, NEP is particularly abundant in the brush border membranes of the kidney and intestine, the lymph nodes and the placenta, and is found in lower concentrations in many other tissues including the vascular wall of the aorta. By finding that the common acute lymphoblastic leukemia antigen was NEP, it was also shown in the state of the art that the enzyme is transiently present at the surface of lymphohaematopoietic cells and elevated levels are found on mature lymphocytes in certain disease states. The clinical interest in NEP, in particular the interest in NEP inhibitors as potential clinical agents derives from the actions of NEP, in conjunction with another zinc metallo-protease, the aminopeptidase N (APN, membrane alanyl aminopeptidase, EC 3.4.11.2), in degrading the enkephalins and also from its role in degrading atrial natriuretic peptide (ANP). For example, it is known that dual inhibitors of NEP and angiotensin converting enzyme (ACE) are potent antihypertensives, resulting from simultaneously increasing the circulating levels of atrial natriuretic peptide, due to NEP inhibition, and decreasing the circulating levels of angiotensin II, due to ACE inhibition. Further interest in the clinical potential of NEP inhibitors came when the peripheral enzyme was shown to degrade the circulating natriuretic and diuretic peptide, atrial natriuretic peptide. NEP inhibitors were therefore investigated for their antihypertensive properties. From a further example it is known that inhibition of enkephalin metabolism by the synthetic NEP inhibitor, thiorphan, gave naloxone-reversible antinociceptive responses in mice. This opened the possibility that, by increasing the levels of endogenous opioids in the regions of their target receptors, an analgesia could be obtained relatively free of the side-effects of morphine or other classical opiate drugs. It was realized that in order to achieve any significant effect, other enkephalin-metabolizing enzymes also had to be inhibited, in particular the aminopeptidase N (APN). Such dual NEP/APN inhibitors completely block enkephalin metabolism and have strong antinociceptive properties.

Endothelin Converting Enzyme (ECE) catalyses the final step in the biosynthesis of the potent vasoconstrictor peptide endothelin (ET). This involves cleavage of the Trp-Val bond in the inactive intermediate, big-endothelin. ECE-1 is a zinc metalloprotease which is homologous with neutral endopeptidase (NEP; neprilysin; EC 3.4.24.11, see above). Like NEP, ECE-1 is inhibited by the compound phosphoramidon and is a type II integral membrane protein. Unlike NEP, however, ECE-1 exists as a disulfide-linked dimer and is not inhibited by other NEP inhibitors such as thiorphan. Immunocytochemical studies indicate a predominant cell-surface location for ECE-1 where it exists as an ectoenzyme. ECE-1 is localized to endothelial cells and some secretory cells, e.g. β-cells in the pancreas, and in smooth muscle cells. Potent and selective inhibitors of ECE, or dual inhibitors of ECE and NEP, may have therapeutic applications in cardiovascular and renal medicine. Endothelin (ET) which is a 21 amino acid bicyclic peptide containing two intramolecular disulfide bonds, is one of the most potent vasoconstricting peptides identified to date and administration to animals results in a sustained increase in blood pressure emphasizing its potential role in cardiovascular regulation. The endogenous production of ET-1 in humans contributes to the maintenance of basal vascular tone. The endothelin system and related enzymes like ECE therefore represent a likely candidate for the development of novel pharmaceutical agents. Thus, the clinical interest in ECE, in particular the interest in ECE inhibitors as potential clinical agents derives from the actions of ECE, in particular in the context of the biosynthesis of ET. Consequently, compounds showing a significant endothelin converting enzyme inhibitory activity are useful in treating and preventing various diseases which are induced or suspected to be induced by ET.

Particular substrates of metalloproteases or metalloendopeptidases known in the state of the art are e.g. big-endothelin-1 (big-ET-1), atrial natriuretic peptides (ANP), and bradykinin. For example, big-ET-1 is known to be a biologically inactive precursor of endothelin-1 (ET-1) which is a highly potent vasoconstrictor peptide that is produced from its precursor big-endothelin-1 via a specific proteolytic processing. ET-1 has a physiological role in the maintenance of basal vascular tone in humans but also seems to be a causative factor in the pathogenesis of various cardiovascular diseases like hypertension, heart failure and atherosclerosis. One approach to attenuate the adverse effects of ET-1 excess is to inhibit the enzymatic conversion of big-ET-1 to ET-1. Since endothelin converting enzyme-1 (ECE-1) was cloned in 1994 (Xu D. et al., Cell, 1994, 78: 473-485), this enzyme has become generally accepted as the endopeptidase responsible for the physiological conversion of big-ET-1 to ET-1. Also since that time, the NEP-inhibitor phosphoramidon became widely accepted as the tool compound which also potently inhibits ECE-1; furthermore, its activity could be verified in heart failure patients given an infusion of big-ET-1 (Love M P et al, Circ, 1996, 94: 2131-2137).

However, despite the fact that ECE-1 has the ability to cleave big-ET-1, more recent reports raise doubts as to whether ECE-1 is the physiologically relevant endothelin converting enzyme, or at least argue that additional enzymes must be involved in the production of ET-1 (Barker S. et al., Mol Pharmacol, 2001, 59: 163-169). Furthermore, according to Barker et al. endothelin-1 (ET-1) has been implicated as a causative factor in the pathogenesis of hypertension, pulmonary hypertension, congestive heart failure, atherosclerosis, and asthma (see also Douglas, 1997, Trends Pharmacol Sci 18:408-412; Haynes and Web, 1998, J Hypertension 16:1081-1098; Goldie and Henry, 1999, Life Sci 65:1-15). A number of highly potent ET receptor antagonist have been reported for therapeutic use, but these compounds are generally selective for ET_{A} receptors or non-selective ET_{A}/ET_{B} antagonists (Douglas, 1997, supra). Although ET_{B} receptors predominate in some tissues, yet they are resistant to blockade by selective ET_{B} or non-selective ET_{A}/ET_{B} antagonists (Hay et al., 1998, J Pharmacol Exp Ther 284:669-677). Therefore, specific inhibition of ET-1 synthesis with ECE inhibitors may be a better approach for attenuating the adverse effects of ET-1 excess under some conditions.

### SUMMARY OF THE INVENTION

It is an object of the present invention to generate new therapeutics for treating and/or inhibiting metalloprotease-related diseases, e.g. by providing therapeutically useful compounds or treatment methods either inhibiting specifically an individual metalloprotease of pharmaceutical interest or specifically inhibiting a selected combination of at least two types of metalloproteases by a combined mode of action profile; and also by providing therapeutically useful combinations of said metalloprotease inhibiting compounds.

In view of the doubts in the state of the art (Barker S. et al., Mol Pharmacol, 2001, 59: 163-169) as to whether ECE-1 is the only physiologically relevant endothelin converting enzyme, and of the conclusion that additional enzymes must be involved in the production of ET-1, according to the present invention a homology cloning project was performed in order to investigate if so far unknown metalloproteases may play a role in the conversion of big-ET-1 to ET-1, and whether specific inhibitors to the newly identified metalloprotease may inhibit this conversion. These efforts resulted in the discovery of a new human gene with high homology to NEP (54% identity) and a somewhat lower homology to ECE-1 (37% identity) which are the two best characterized members of the neprilysin metalloprotease family. The polypeptide product of this human gene was found to be abundantly expressed in a number of human tissues, and was designated the working title “IGS5” (see co-pending international patent application PCT/EP 00/11532).

Therefore, generally the present invention pertains to the use of a compound having combined, in particular by concurrent, inhibitory activity

a) on neutral endopeptidase (NEP) and

b) the metalloprotease IGS5 which is a polypeptide comprising an amino acid sequence which has at least 70% identity to one of the amino acid sequences selected from the group of SEQ ID NO:2, SEQ ID NO:4 and SEQ ID NO:6;

or of a pharmaceutically acceptable salt or solvate or biolabile ester thereof, for the manufacture of a pharmaceutical composition, and for related treatment methods, for treating a mammal, preferably a human, suffering from or being susceptible to a disease or condition which can be alleviated or prevented by combined, in particular by concurrent, inhibition of NEP and IGS5.

In a particular aspect the present invention pertains to the use of a compound with combined NEP/IGS5 inhibitory activity, or a pharmaceutically acceptable salt or solvate or biolabile ester thereof, for the manufacture of a pharmaceutical composition, and for related treatment methods, for treating a mammal, preferably a human, suffering from or being susceptible to a disease or to a condition where big-ET-1 levels are elevated and which disease (condition) can be alleviated or prevented by combined, in particular concurrent, inhibition of NEP and IGS5.

In a further particular aspect the present invention pertains to the use of a compound with combined NEP/IGS5 inhibitory activity, or a pharmaceutically acceptable salt or solvate or biolabile ester thereof, for the manufacture of a pharmaceutical composition, and for related treatment methods, for treating a mammal, preferably a human, suffering from or being susceptible to a disease or condition where ET-1 is significantly upregulated and which disease (condition) can be alleviated or prevented by combined, in particular concurrent, inhibition of NEP and IGS5.

In a further particular aspect the present invention pertains to the use of said compounds with combined or concurrent NEP/IGS5 inhibitory activity or a pharmaceutically acceptable salt or solvate or biolabile ester thereof for the manufacture of a pharmaceutical composition, and for related treatment methods, preferably for treatment and/or prohylaxis of hypertension, including secondary forms of hypertension such as renal or pulmonary hypertension, heart failure, angina pectoris, arrhythmias, myocardial infarction, cardiac hypertrophy, cerebral ischemia, peripheral vascular disease, subarachnoidal hemorrhage, chronic obstructive pulmonary disease (COPD), asthma, renal disease, atherosclerosis, and pain in colorectal cancer or prostate cancer, in larger mammals, preferably in humans.

Furthermore, it may be beneficial to combine these compounds showing combined or concurrent NEP/IGS5 inhibitory activity with individual and/or combined metalloprotease inhibitors other than the NEP/IGS5 inhibitors, e.g. with separate ACE- and/or ECE- and/or NEP-inhibitors and/or mixed inhibitors of these metalloproteases.

### DETAILED DESCRIPTION OF THE INVENTION

Definitions

The terms used in the present application, unless explicitly defined otherwise hereinafter, have the meaning as usually understood by the skilled artisan in the field of this invention. The following definitions are provided to facilitate understanding of certain terms used frequently herein.

“IGS5” refers, among others, to a polypeptide comprising the amino acid sequence set forth in one of SEQ ID NO:2, SEQ ID NO:4 and SEQ ID NO:6, or respective variants thereof. Thus “IGS5” particularly includes IGS5PROT, IGS5PROT1 and IGS5PROT2.

“Enzyme Activity” or “Biological Activity” refers to the metabolic or physiologic function of said IGS5 including similar activities or improved activities or these activities with decreased undesirable side effects.

“IGS5-gene” refers to a polynucleotide comprising the nucleotide sequence set forth in one of SEQ ID NO:1, SEQ ID NO:3 and SEQ ID NO:5, or respective variants, e.g. allelic variants, thereof and/or their complements.

“Identity”, as known as a measure of identity in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences, e.g. in generally by alignment of the sequences so that the highest order match is obtained. Thus “Identity” and or the alternative wording “Similarity” has an art-recognized meaning and can be readily calculated by known methods, including but not limited to those described in “Computational Molecular Biology”, Lesk, A. M., Ed., Oxford University Press, New York, 1988; “Biocomputing: Informatics and Genome Projects”, Smith, D. W., Ed., Academic Press, New York, 1993; “Computer Analysis of Sequence Data”, Part I, Griffin, A. M., and Griffin, H. G., Eds., Humana Press, New Jersey, 1994; “Sequence Analysis in Molecular Biology”, von Heinje, G., Academic Press, 1987; “Sequence Analysis Primer”, Gribskov, M. and Devereux, J., Eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990)). The well known Smith Waterman algorithm may also be used to determine identity. A publicly available program useful to determine identity or similarity of polypeptide sequences or polynucleotide sequence, respectively, is known as the “gap” program from Genetics Computer Group, Madison Wis., which is usually run with the default parameters for comparisons (along with no penalty for end gaps). The preferred (i.e. default) parameters for polypeptide sequence comparison include the following: Algorithm as described by Needleman and Wunsch, J. Mol. Biol. 48: 443-453 (1970); Comparison Matrix BLOSSUM62 from Hentikoff and Hentikoff, Proc. Natl. Acad. Sci. USA. 89:10915-10919 (1992); Gap Penalty: 12; Gap Length Penalty: 14. The preferred (i.e. default) parameters for polynucleotide sequence comparison include the following: Algorithm as described by Needleman and Wunsch, J. Mol. Biol. 48: 443-453 (1970); Comparison Matrix: matches=+10, mismatch=0; Gap Penalty: 50; Gap Length Penalty: 3. The word “homology” may substitute for the word “identity”.

As an illustration, by a polynucleotide having a nucleotide sequence having at least, for example, 95% “identity” to a reference nucleotide sequence, for example to a reference nucleotid sequence selected from the group of SEQ ID NO:1, SEQ ID NO:3 and SEQ ID NO:5, is intended that the nucleotide sequence of the polynucleotide is identical to the reference sequence except that the polynucleotide sequence may include up to five point mutations per each 100 nucleotides of the respective reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence, or in a number of nucleotides of up to 5% of the total nucleotides in the reference sequence there may be a combination of deletion, insertion and substitution. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

Similarly, by a polypeptide having an amino acid sequence having at least, for example 95% “identity” to a reference amino acid sequence, for example to a reference amino acid sequence selected from the group of SEQ ID NO:2, SEQ ID NO:4 and SEQ ID NO:6, is intended that the amino acid sequence of the polypeptide is identical to the reference sequence except that the polypeptide sequence may include up to five amino acid alterations per each 100 amino acids of the respective reference amino acid. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

“Homolog” is a generic term used in the art to indicate a polynucleotide or polypeptide sequence possessing a high degree of sequence relatedness to a subject sequence. Such relatedness may be quantified by determining the degree of identity and/or similarity between the sequences being compared as herein described. Falling within this generic term are the terms “ortholog”, meaning a polynucleotide or polypeptide that is the functional equivalent of a polynucleotide or polypeptide in another species, and “paralog” meaning a functionally similar sequence when considered within the same species. Hence, in humans for example, within the family of endothelin converting enzymes ECE-1 is a paralog of the other members, e.g. of ECE-2.

Pharmacological Evaluation of the Newly identified Metalloprotease IGS5 of Specific Inhibitors to this Metalloprotease

In view of the doubts in the state of the art (Barker S. et al., Mol Pharmacol, 2001, 59: 163-169) as to whether ECE-1 is the only physiologically relevant endothelin converting enzyme, and of the conclusion that additional enzymes must be involved in the production of ET-1, according to the present invention a homology cloning project was performed in order to investigate if so far unknown metalloproteases may play a role in the conversion of big-ET-1 to ET-1, and whether specific inhibitors to the newly identified metalloprotease may inhibit this conversion. Under the assumption that enzymes with similar activity should have similarities in their amino acid sequences, the human genome was searched for DNA sequences coding for proteins with homology to NEP and ECE, the two best characterized members of the neprilysin metalloprotease family. These efforts resulted in the discovery of a new human gene with high homology to NEP (54% identity) and a somewhat lower homology to ECE-1 (37% identity). The polypeptide product of this gene was found to be abundantly expressed in a number of human tissues, and designated the working title “IGS5”. The cloning, expression and basic biological characterization of IGS5 is described in detail in the co-pending international patent application PCT/EP 00/11532 which entire content is particularly incorporated by reference to further illustrate the respective details given below on IGS5.

In order to characterize and evaluate the pharmacological enzymatic properties of IGS5 for the purpose of the present invention a human IGS5 protein was generated by using an insect cell line as the expression system, and a variety of potential substrates of the IGS5 protein were tested. IGS5 was confirmed to efficiently cleave big-ET-1, bradykinin and substance P, thus further confirming that this novel protein is a genuine metalloprotease with a broad substrate specificity, which is a common feature of metalloproteases and which feature has been reported for NEP, ECE-1 and also ACE. It should also be noted that according to the findings of the present invention the proteolysis of big-ET-1 by IGS5 surprisingly results in the correct formation of ET-1, e.g. big-ET-1 is correctly cleaved between amino acids Trp21 and Val22.

Furthermore, according to the present invention for the first time the potency of metalloprotease inhibitor compounds to suppress the conversion of big-ET to ET-1 was examined, using a labeled fluorescent big-ET-1 analog. The results are summarized in Table 9 of the experimental section. It is of interest that phosphoramidon that is known to inhibit the conversion of big-ET to ET-1 in vivo, also inhibits IGS5 with high potency in the biochemical assay used in the present invention, and surprisingly that the inhibition of IGS5 by phosphoramidon is actually considerably higher than ECE-1. In contrast, the selective NEP inhibitor thiorphan as well as the selective ECE-1 inhibitor SM-19712 (4-chloro-N-[[(4-cyano-3-methyl-1-phenyl-1H-pyrazol-5-yl)amino]carbonyl]benzenesulfonamide, monosodium salt; Umekawa K, Hasegawa H, Tsutsumi Y, Sato K, Matsumura Y, Ohashi N., J Pharmacol 2000 September; 84(1):7-15; Discovery Research Laboratories I, Research Center, Sumitomo Pharmaceuticals Co, Ltd, Osaka, Japan) do not affect the activity of IGS5 (Table 9, see experimental section).

A very particular aspect of the present invention is the most important and unique finding that-numerous compounds which revealed to be metalloprotease inhibitors are able to inhibit IGS5 enzyme even at low nanomolar concentrations, e.g. at concentrations corresponding to IC₅₀ values in the range of about 1 to 10 nM, and thus prove to also specifically inhibit the newly identified IGS5 metalloprotease of particular pharmaceutical interest.

Therefore, generally the present invention pertains to the use of a compound having combined, in particular concurrent, inhibitory activity

a) on neutral endopeptidase (NEP) and

b) on the metalloprotease IGS5 which is a polypeptide comprising an amino acid sequence which has at least 70% identity to one of the amino acid sequences selected from the group of SEQ ID NO:2, SEQ ID NO:4 and SEQ ID NO:6;

or of a pharmaceutically acceptable salt or solvate or biolabile ester thereof, for the manufacture of a medicament (pharmaceutical composition) for treating a mammal, preferably a human, suffering from or being susceptible to a condition which can be alleviated or prevented by combined, in particular concurrent, inhibition of NEP and IGS5.

In a particular aspect the present invention pertains to the use of a compound with combined NEP/IGS5 inhibitory activity, or a pharmaceutically acceptable salt or solvate or biolabile ester thereof, for the manufacture of a medicament (pharmaceutical composition) for treating a mammal, preferably a human, suffering from or being susceptible to a disease or condition where big-ET-1 levels are elevated and which disease or condition can be alleviated or prevented by combined, in particular concurrent, inhibition of NEP and IGS5.

In a further particular aspect the present invention pertains to the use of a compound with combined NEP/IGS5 inhibitory activity, or a pharmaceutically acceptable salt or solvate or biolabile ester thereof, for the manufacture of a medicament (pharmaceutical composition) for treating a mammal, preferably a human, suffering from or being susceptible to a disease or condition where ET-1 is significantly upregulated and which disease or condition can be alleviated or prevented by combined, in particular concurrent, inhibition of NEP and IGS5.

“Combined, or in particular concurrent, inhibitory activity” in the sense of the invention means at least the dual inhibition of NEP and IGS5 by concurrent block of both enzymatic systems, NEP and IGS5, and potentially additional concurrent inhibition of a third system, e.g. triple inhibition of the enzymatic systems NEP, IGS5 and e.g. ECE-1. According to the results of the present invention it may be expected that this combined or concurrent inhibition of both enzymatic systems, NEP and IGS5, is more effective than the isolated blockade of either group by different compounds or just the blockade of each of said individual enzymes. Thus, the present invention provides a new therapeutical concept by suggesting the use of combined, in particular concurrent, NEP and IGS5 inhibitors for the treatment and/or prophylaxis or inhibition of a set of certain diseases or conditions which can be alleviated or prevented by combined, in particular concurrent, inhibition of NEP and IGS5. In this respect the invention also provides compounds which show combined or concurrent inhibition of both, NEP and IGS5, thereby providing a novel and prospective use of compounds with increased therapeutical value for the treatment and/or prophylaxis or inhibition of the concerned diseases or conditions.

Combined, in particular concurrent, mechanisms of action are of outstanding medical interest as therapeutical benefits can be expected which are more pronounced than modulating each singular system separately.

For example, with regard to endothelin-converting enzyme inhibitors further elucidation of current status and perspectives of this principle and the related benefits recently were reported in a review by B.-M. Löffler (J. Cardiovasc. Pharmacol. (2000), 35(Suppl. 2), S79-S82). According to Löffler, recent research has led to the discovery of potent selective or mixed endothelin-converting enzyme (ECE), ECE/neutral endopeptidase (NEP) and ECE/NEP/angiotensin-converting enzyme (ACE) inhibitors. There is also reported increasing evidence, that the functions of the endothelin (ET), renin-angiotensin and NEP systems for the regulation of the cardiovascular homeostasis are connected by a complex regulation network. Thus Löffler estimates that it will be a challenging task of future research with the newly available selective and mixed ECE-1 inhibitors to show whether the combined inhibition of more than one cardiovascular system is superior to selective inhibition. In this respect the present invention provides a superior progress in that for the first time combined or concurrent inhibition of at least the enzymatic systems NEP and IGS5 is contributed to the state of the art.

In this particular aspect the present invention pertains to the use of a compound or a pharmaceutically acceptable salt or solvate or biolabile ester thereof for the manufacture of a medicament (pharmaceutical composition) for treatment and/or prohylaxis of hypertension, including secondary forms of hypertension such as renal or pulmonary hypertension, heart failure, angina pectoris, arrhythmias, myocardial infarction, cardiac hypertrophy, cerebral ischemia, peripheral vascular disease, subarachnoidal hemorrhage, chronic obstructive pulmonary disease (COPD), asthma, renal disease, atherosclerosis, and pain in colorectal cancer or prostate cancer, in mammals, preferably in humans. In particular, in the present invention the compounds with combined or concurrent NEP/IGS5-inhibitory activity preferably are used for the treatment and/or prohylaxis of said diseases or conditions, in a patient sub-population suffering from or being susceptible to a disease or condition which can be alleviated or prevented by combined, in particular concurrent, inhibition of NEP and IGS5.

Furthermore, it may be beneficial to additionally combine the compounds showing combined or concurrent NEP/IGS5 inhibitory activity according to the invention with other individual and/or combined metalloprotease inhibitors than combined NEP/IGS5 inhibitors. Such other metalloprotease inhibitors that may be used in combination with compounds with combined NEP/IGS5 inhibitory activity are for example ACE inhibitors such as captopril, enalapril, lisinopril, fosinopril, perindopril, quinapril, ramipril; furthermore, selective ECE inhibitors such as compound SM-19712 (Sumitomo, supra); selective NEP inhibitors such as thiorphan; dual NEP/ECE inhibitors such as compound CGS-35066 (De Lombart et al., J. Med. Chem. 2000, Feb. 10; 43(3):488-504); or mixed inhibitors of these metalloproteases such as omapatrilat or sampatrilat. By this type of combination treatment and/or prophylaxis or inhibition the therapeutic value of the compounds with combined or concurrent NEP/IGS5 inhibitory activity still may be further increased, in particular with regard to the diseases and/or conditions mentioned above. Therefore in a further aspect the invention also pertains to a combination therapy and/or a combination prophylaxis or inhibition which are further described below.

In particular, according to the present invention it was found that compounds which are primarily neutral endopeptidase inhibitors (NEP-inhibitors) are well suited to inhibit also the newly identified IGS5 metalloprotease enzyme of pharmaceutical interest even at said low nanomolar concentrations, and thus these compounds prove to be metalloprotease inhibitors with a combined mode of action profile, that is e.g. a combined or concurrent selective NEP/IGS5 inhibitory activity profile. Such compounds may have a structure of formula I:

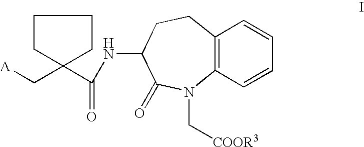

wherein

A stands for a group with formula II or III

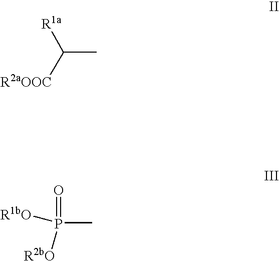

in which formula II

R^{1a} stands for a phenyl-lower-alkyl group which can be optionally substituted in the phenyl ring by lower alkyl, lower alkoxy or halogen, or for a naphthyl-lower-alkyl group,

R^{2a} means hydrogen or a group forming a biolabile ester; and in which formula III

R^{1b} is hydrogen or a group forming a biolabile phosphonic acid ester,

R^{2b} is hydrogen or a group forming a biolabile phosphonic acid ester; and wherein

R³ means hydrogen or a group forming a biolabile carboxylic acid ester; and physiologically acceptable salts of acids or solvates of the formula I.

Where the substituents in the compounds of formula I are or contain lower alkyl or alkoxy groups, these can be straight-chain or branched and contain, in particular, 1 to 4, preferably 1 to 2, carbon atoms and are preferably methyl or methoxy. Where the substituents contain halogen, particularly suitable are fluorine, chlorine or bromine, preferably fluorine or chlorine.

In the radical R^{1a} the lower alkylene chain can contain 1 to 4, preferably 1 to 2, carbon atoms. R^{1a} in particular is an optionally substituted phenethyl group which can optionally be substituted one or more times by halogen, lower alkoxy or lower alkyl, or is a naphthylethyl group.

The compounds of formula Ia are optionally esterified dicarboxylic acid derivatives.

Suitable groups R³ forming biolabile carboxylic acid esters are those which can be cleaved under physiological conditions in vivo with release of the carboxylic acid. For example, those suitable for this purpose are lower alkyl groups, phenyl or phenyl-lower alkyl groups optionally mono- or polysubstituted in the phenyl ring by lower alkyl or lower alkoxy or by a lower alkylene chain bonded to two adjacent carbon atoms, dioxolanylmethyl groups optionally substituted in the dioxolane ring by lower alkyl or C₂-C₆-alkanoyloxymethyl groups optionally substituted on the oxymethyl group by lower alkyl. If the group R³ forming a biolabile ester is or contains lower alkyl, this can be branched or unbranched and can contain 1 to 4 carbon atoms. If the group forming a biolabile ester is an optionally substituted phenyl-lower alkyl group, this can contain an alkylene chain having 1 to 3, preferably 1, carbon atom(s) and is preferably benzyl. If the phenyl ring is substituted by a lower alkylene chain, this can contain 3 to 4, preferably 3, carbon atoms. If R³ is an optionally substituted alkanoyloxymethyl group, this can contain a preferably branched alkanoyloxy group having 2 to 6, preferably 3 to 5, carbon atoms and can be, for example, a pivaloyloxymethyl radical (=tert-butylcarbonyl-oxymethyl radical).

Suitable groups R^{2a} forming biolabile carboxylic acid esters are those which can be cleaved under physiological conditions in vivo with release of the carboxylic acid, and correspond to the groups exemplified for group R³ supra.

Groups R^{1b} and R^{2b} suitable as groups forming biolabile phosphonic acid esters are those which can be removed under physiological conditions in vivo with release of the respective phosphonic acid function. For example, groups which are suitable for this purpose are lower alkyl groups, C₂-C₆-alkanoyloxymethyl groups optionally substituted on the oxymethyl group by lower alkyl, or phenyl or phenyl-lower alkyl groups whose phenyl ring is optionally mono- or polysubstituted by lower alkyl, lower alkoxy or by a lower alkylene chain bonded to two adjacent carbon atoms. If the group R^{1b} and/or R^{2b} forming a biolabile ester is or contains lower alkyl, this can be branched or unbranched and can contain 1 to 4 carbon atoms. If R^{1b} and/or R^{2b} are an optionally substituted alkanoyloxymethyl group, it can contain a preferably branched alkanoyloxy group having 2 to 6, preferably 3 to 5, carbon atoms and can, for example, be a pivaloyloxymethyl radical (=tert-butylcarbonyloxymethyl radical). If R^{1b} and/or R^{2b} are an optionally substituted phenyl-lower alkyl group, this can contain an alkylene chain having 1 to 3, preferably 1, carbon atoms. If the phenyl ring is substituted by a lower alkylene chain, this can contain 3 to 4, in particular 3, carbon atoms and the substituted phenyl ring is in particular indanyl.

Suitable physiologically acceptable salts of acids of formula I include their alkali metal, alkaline earth metal or ammonium salts, for example sodium, potassium or calcium salts or salts with physiologically acceptable, pharmacologically neutral organic amines such as, for example, diethylamine or tert-butylamine, or phenyl-lower alkylamines such as α-methylbenzylamine.

The compounds of the formula I contain at least one chiral carbon atom, namely the carbon atom carrying the amide side chain in the 3-position of the benzazepine structure. The compounds can thus be present in two optically active stereoisomeric forms or as a racemate. The present invention includes both the racemic mixtures and the isomerically pure compounds of the formula I. If in the compounds of the formula I the group A stands for formula II, the compounds of formula I contain two chiral carbon atoms, namely the carbon atom which is in position 3 of the ring framework and carries the amide side-chain, and the carbon atom of the amide side-chain which carries the radical R^{1a}. These compounds of the formula I, in which the group A stands for formula II, can therefore exist in several optically active stereoisomeric forms or as a racemate. According to the present invention both the racemic mixtures and the isomerically pure compounds may be used. If in the compounds of the formula I the group A stands for formula III, and R^{1b} and R^{2b} are not hydrogen and in each case have different meanings, the phosphorus atom of the phosphonic acid group can also be chiral. The invention also relates to the isomer mixtures and isomerically pure compounds of the formula I, in which the group A stands for formula III, formed as a result of chiral phosphorus atoms.

According to the invention, the compounds of the formula I, their salts and biolabile esters may be obtained in a manner known per se in the state of the art (see below).

In preferred embodiment of the present invention it was found that primariliy NEP-inhibitory compounds such as benzazepinone-N-acetic acid derivatives with structure of formula Ia, or such as phosphono-substituted benzazepinone derivatives of structure of formula Ib.

Compounds with structure of formula Ia are already known as NEP-inhibiting compounds from U.S. Pat. No. 5,677,297 said compounds being useful for the treatment of diseases or conditions as referenced supra. The compounds of the formula Ia have the following structure

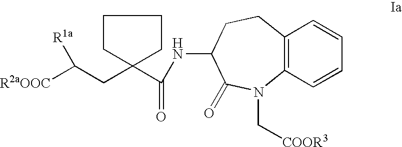

wherein

R^{1a} stands for a phenyl-lower-alkyl group which can be optionally substituted in the phenyl ring by lower alkyl, lower alkoxy or halogen, or for a naphthyl-lower-alkyl group,

R^{2a} means hydrogen or a group forming a biolabile ester and

R³ means hydrogen or a group forming a biolabile ester, and physiologically acceptable salts of acids of the formula I.

Where the substituents in the compounds of formula Ia are or contain lower alkyl or alkoxy groups, these can be straight-chain or branched and contain, in particular, 1 to 4, preferably 1 to 2, carbon atoms and are preferably methyl or methoxy. Where the substituents contain halogen, particularly suitable are fluorine, chlorine or bromine, preferably fluorine or chlorine.

In the radical R^{1a} the lower alkylene chain can contain 1 to 4, preferably 1 to 2, carbon atoms. R^{1a} in particular is an optionally substituted phenethyl group which can optionally be substituted one or more times by halogen, lower alkoxy or lower alkyl, or is a naphthylethyl group.

The compounds of formula Ia are optionally esterified dicarboxylic acid derivatives. Depending on the mode of administration, biolabile monoesters, particularly compounds in which R^{2a} is a group forming a biolabile ester and R³ is hydrogen, or dicarboxylic acids are preferred, the latter being particularly suitable for i.v. administration.

Suitable R^{2a} and R³ groups, in compounds of formula Ia, forming biolabile esters are lower alkyl groups, phenyl or phenyl-lower-alkyl groups which are optionally substituted in the phenyl ring by lower alkyl or by a lower alkylene chain bonded to two adjacent carbon atoms, dioxolanylmethyl groups which are optionally substituted in the dioxolane ring by lower alkyl, or C₂-C₆-alkano-yloxymethyl groups optionally substituted on the oxymethyl group by lower alkyl. Where the R^{2a} or R³ group forming a biolabile ester is lower alkyl, this can be a preferably unbranched alkyl group with 1 to 4, preferably 2, carbon atoms. Where the group forming a biolabile ester is an optionally substituted phenyl-lower-alkyl group, its alkylene chain can contain 1 to 3, preferably 1, carbon atom. Where the phenyl ring is substituted by a lower alkylene chain, this can contain 3 to 4, particularly 3, carbon atoms. Phenyl, benzyl or indanyl are particularly suitable as phenyl-containing substituents R^{2a} and/or R³. Where R^{2a} and/or R³ are an optionally substituted alkanoyloxymethyl group, their alkanoyloxy group can contain 2 to 6, preferably 3 to 5, carbon atoms and is preferably branched and can be, for example, a pivaloyloxymethyl radical (=tert-butylcarbonyl-oxymethyl radical).

Suitable physiologically acceptable salts of dicarboxylic acids or monoesters of formula I include their alkali metal, alkaline earth metal or ammonium salts, for example sodium or calcium salts or salts with physiologically acceptable, pharmacologically neutral organic amines such as, for example, diethylamine or tert-butylamine.

The compounds of formula Ia contain two chiral carbon atoms, namely the carbon atom which is in position 3 of the ring framework and carries the amide side-chain, and the carbon atom of the amide side-chain which carries the radical R^{1a}. The compounds can therefore exist in several optically active stereoisomeric forms or as a racemate. According to the present invention both the racemic mixtures and the isomerically pure compounds of formula Ia may be used.

According to the invention, the compounds of the formula Ia and their salts and biolabile esters may be obtained in a manner known per se in the state of the art, e.g. as described in U.S. Pat. No. 5,677,297.

Preferred compounds of the formula Ia e.g. those in which R² and/or R³ means a group forming a biolabile ester, and physiologically acceptable salts thereof. The groups forming a biolabile ester may be a lower alkyl group, or a phenyl or phenyl-lower-alkyl group, particularly phenyl, benzyl or indanyl, which is optionally substituted in the phenyl ring by lower alkyl or by a lower alkylene chain bonded to two adjacent carbon atoms, or a dioxolanylmethyl group, particularly (2,2-dimethyl-1,3-dioxolane-4-yl)methyl, which is optionally substituted in the dioxolane ring by lower alkyl, or a C₂-C₆-alkanoyloxymethyl group optionally substituted on the oxymethyl group by lower alkyl. In particular compounds of formula Ia are preferred which are characterized in that R² is a group forming a biolabile ester and R³ is hydrogen.

Particular preferred examples of compounds of formular Ia are, e.g.

(3S,2′R)-3-{1-[2′-carboxy-4′-phenylbutyl]-cyclopentane-1-carbonylamino}-2,3,4,5-tetrahydro-2-oxo-1H-1-benzazepine-1-acetic acid, (compound Ia-1);

(3S,2′R)-3-{1-[2′-(ethoxycarbonyl)-4′-phenylbutyl]-cyclopentane-1-carbonylamino}-2,3,4,5-tetrahydro-2-oxo-1H-1-benzazepine-1-acetic acid, (compound Ia-2);

(3S,2′R)-3-{1-[2′-(ethoxycarbonyl)-4′-naphthylbutyl]-cyclopentane-1-carbonylamino}-2,3,4,5-tetrahydro-2-oxo-1H-1-benzazepine-1-acetic acid, (compound Ia-3);

and physiologically acceptable salts of acids or solvates thereof.

Further particular examples of compounds of formular Ia are, e.g.

3-{1-[2′-(ethoxycarbonyl)-4′-phenylbutyl]-cyclopentane-1-carbonylamino}-2,3,4,5-tetrahydro-2-oxo-1H-1-benzazepine-1-acetate-tert-butylester, (compound Ia-4);

3-{1-[2′-(ethoxycarbonyl)-4′-phenylbutyl]cyclopentane-1-carbonylamino}-2,3,4,5-tetrahydro-2-oxo-1H-1-benzazepine-1-acetic acid, (compound Ia-5);

(3S,2′R)-3-{1-[2′-ethoxycarbonyl)-4′-phenylbutyl]cyclopentane-1-carbonylamino}-2,3,4,5-tetrahydro-2-oxo-1H-1-benzazepine-1-acetate-tert-butylester, (compound Ia-6);

(3S,2′R)-3-{1-[2′-(carboxy-4′-phenylbutyl]cyclopentane-1-carbonylamino}-2,3,4,5-tetrahydro-2-oxo-1H-1-benzazepine-1-acetic acid, (compound Ia-7);

3-{1-[2′-(tert-butoxycarbonyl)-4′-phenylbutyl]cyclopentane-1-carbonylamino}-2,3,4,5-tetrahydro-2-oxo-1H-1-benzazepine-1-acetate-tert-butylester, (compound Ia-8);

3-[1-(2′-carboxy-4′-phenylbutyl)cyclopentane-1-carbonylamino]-2,3,4,5-tetrahydro-2-oxo-1H-1-benzazepine-1-acetic acid, (compound Ia-9);

3-{1-[2′-(tert-butoxycarbonyl)-4′-phenylbutyl]cyclopentane-1-carbonylamino}-2,3,4,5-tetrahydro-2-oxo-1H-1-benzazepine-1-acetate-benzylester, (compound Ia-10);

3-[1-(2′-carboxy-4′-phenylbutyl)cyclopentane-1-carbonylamino]-2,3,4,5-tetrahydro-2-oxo-1H-1-benzazepine-1-acetate-benzylester, (compound Ia-11);

3-{1-[2′-(tert-butylcarbonyloxymethoxycarbonyl)-4′-phenylbutyl]cyclopentane-1-carbonylamino}-2,3,4,5-tetrahydro-2-oxo-1H-1-benzazepine-1-acetate-benzylester, (compound Ia-12);

3-{1-[2′-(pivaloyloxymethoxycarbonyl)-4′-phenylbutyl]-cyclopentane-1-carbonylamino}-2,3,4,5-tetrahydro-2-oxo-1H-1-benzazepine-1-acetic acid, (compound Ia-13);

and physiologically acceptable salts of acids or solvates thereof.

Compounds with structure of formula Ib are already known as NEP-inhibiting compounds and in addition slightly endothelin converting enzyme inhibitors (ECE-inhibitors) from U.S. Pat. No. 5,952,327, said compounds being useful for the treatment of the diseases or conditions referenced supra. Thus, the invention also relates to compounds of the formula Ib

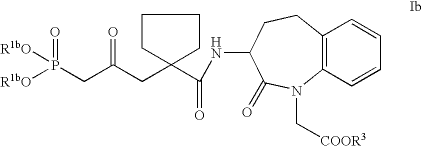

wherein

R^{1b} is hydrogen or a group forming a biolabile phosphonic acid ester,

R^{2b} is hydrogen or a group forming a biolabile phosphonic acid ester and

R³ is hydrogen or a group forming a biolabile carboxylic acid ester and physiologically acceptable salts of acids of the formula Ib.

The compounds of the formula Ib are acid derivatives comprising carboxylic acid and phosphonic acid groups which are optionally esterified by groups forming biolabile esters. The biolabile esters of the formula Ib are prodrugs of the free acids. Depending on the administration form, the biolabile esters or the acids are preferred, the latter in particular being suitable for i.v. administration.

Groups R^{1b} and R^{2b} suitable as groups forming biolabile phosphonic acid esters are those which can be removed under physiological conditions in vivo with release of the respective phosphonic acid function. For example, groups which are suitable for this purpose are lower alkyl groups, C₂-C₆-alkanoyloxymethyl groups optionally substituted on the oxymethyl group by lower alkyl, or phenyl or phenyl-lower alkyl groups whose phenyl ring is optionally mono- or polysubstituted by lower alkyl, lower alkoxy or by a lower alkylene chain bonded to two adjacent carbon atoms. If the group R^{1b} and/or R^{2b} forming a biolabile ester is or contains lower alkyl, this can be branched or unbranched and can contain 1 to 4 carbon atoms. If R^{1b} and/or R^{2b} are an optionally substituted alkanoyloxymethyl group, it can contain a preferably branched alkanoyloxy group having 2 to 6, preferably 3 to 5, carbon atoms and can, for example, be a pivaloyloxymethyl radical (=tert-butylcarbonyloxymethyl radical). If R^{1b} and/or R^{2b} are an optionally substituted phenyl-lower alkyl group, this can contain an alkylene chain having 1 to 3, preferably 1, carbon atoms. If the phenyl ring is substituted by a lower alkylene chain, this can contain 3 to 4, in particular 3, carbon atoms and the substituted phenyl ring is in particular indanyl.

Suitable groups R³ for compounds of formula Ib forming biolabile carboxylic acid esters are those which can be cleaved under physiological conditions in vivo with release of the carboxylic acid. For example, those suitable for this purpose are lower alkyl groups, phenyl or phenyl-lower alkyl groups optionally mono- or polysubstituted in the phenyl ring by lower alkyl or lower alkoxy or by a lower alkylene chain bonded to two adjacent carbon atoms, dioxolanylmethyl groups optionally substituted in the dioxolane ring by lower alkyl or C₂-C₆-alkanoyloxymethyl groups optionally substituted on the oxymethyl group by lower alkyl. If the group R³ forming a biolabile ester is or contains lower alkyl, this can be branched or unbranched and can contain 1 to 4 carbon atoms. If the group forming a biolabile ester is an optionally substituted phenyl-lower alkyl group, this can contain an alkylene chain having 1 to 3, preferably 1, carbon atom(s) and is preferably benzyl. If the phenyl ring is substituted by a lower alkylene chain, this can contain 3 to 4, preferably 3, carbon atoms. If R³ is an optionally substituted alkanoyloxymethyl group, this can contain a preferably branched alkanoyloxy group having 2 to 6, preferably 3 to 5, carbon atoms and can be, for example, a pivaloyloxymethyl radical.

According to the invention, the compounds of the formula Ib and their salts and biolabile esters may be obtained in a manner known in the state of the art.

Suitable physiologically acceptable salts of acids of the formula Ib are in each case their alkali metal, alkaline earth metal or ammonium salts, for example their sodium, potassium or calcium salts or salts with physiologically acceptable, pharmacologically neutral organic amines such as, for example, diethylamine, tert-butylamine or phenyl-lower alkylamines such as α-methylbenzylamine.

The compounds of the formula Ib contain a chiral carbon atom, namely the carbon atom carrying the amide side chain in the 3-position of the benzazepine structure. The compounds can thus be present in two optically active stereoisomeric forms or as a racemate. The present invention includes both the racemic mixtures and the isomerically pure compounds of the formula I. If R^{1b} and R^{2b} in compounds of the formula Ib are not hydrogen and in each case have different meanings, the phosphorus atom of the phosphonic acid group can also be chiral. The invention also relates to the isomer mixtures and isomerically pure compounds of the formula I formed as a result of chiral phosphorus atoms.

Preferred compound of formula Ib are those, in which R³ stands for hydrogen or lower alkyl, e.g. C₁-C₄-alkyl, in particular C₁-C₂-alkyl, and physiologically acceptable salts of acids of the formula Ib.

Particular examples of compounds of formular Ib are, e.g.

Benzyl (3S)-3-(1-dibenzylphosphonomethyl-cyclopentane-1-carbonylamino)-2,3,4,5-tetrahydro-2-oxo-1H-1-benzazepine-1-acetate, (=compound Ib-1);

(3S)-3-(1-Phosphonomethyl-cyclopentane-1-carbonylamino)-2,3,4,5-tetrahydro-2-oxo-1H-1-benzazepine-1-acetic acid, (=compound Ib-2);

Benzyl (3S)-3-(1-benzylethylphosphonomethyl-cyclopentane-1-carbonylamino)-2,3,4,5-tetrahydro-2-oxo-1H-1-benzazepine-1-acetate, (=compound Ib-3);

Ethyl (3S)-3-(1-benzylethylphosphonomethyl-cyclopentane-1-carbonylamino)-2,3,4,5-tetrahydro-2-oxo-1H-1-benzazepine-1-acetate, (=compound Ib-4);

Ethyl (3S)-3-(1-ethylphosphonomethyl-cyclopentane-1-carbonylamino)-2,3,4,5-tetrahydro-2-oxo-1H-1-benzazepine-1-acetate, (=compound Ib-5);

Ethyl (3S)-3-[1-(pivaloyloxymethylethylphosphonomethyl)-cyclopentane-1-carbonylamino]-2,3,4,5-tetrahydro-2-oxo-1H-benzazepine-1-acetate, (=compound Ib-6);

Ethyl (3S)-3-[1-(5-indanylethylphosphonomethyl)-cyclopentane-1-carbonylamino]-2,3,4,5-tetrahydro-2-oxo-1H-benzazepine-1-acetate, (=compound Ib-7);

tert-Butyl (3S)-3-(1-benzylethylphosphonomethyl-cyclopentane-1-carbonylamino)-2,3,4,5-tetrahydro-2-oxo-1H-1-benzazepine-1-acetate, (=compound Ib-8);

Benzyl (3S)-3-(1-ethylphosphonomethyl-cyclopentane-1-carbonylamino)-2,3,4,5-tetrahydro-2-oxo-1H-benzazepine-1-acetate, (=compound Ib-9);

Benzyl (3S)-3-(1-diethylphosphonomethyl-1-cyclopentane-1-carbonylamino)-2,3,4,5-tetrahydro-2-oxo-1H-benzazepine-1-acetate, (=compound Ib-10);

(3S)-3-(1-Diethylphosphonomethyl-cyclopentane-1-carbonylamino)-2,3,4,5-tetrahydro-2-oxo-1H-1-benzazepine-1-acetic acid, (=compound Ib-11);

Ethyl (3S)-3-(1-diethylphosphonomethyl-cyclopentane-1-carbonylamino)-2,3,4,5-tetrahydro-2-oxo-1H-1-benzazepine-1-acetate, (=compound Ib-12);

Ethyl (3S)-3-(1-phosphonomethyl-cyclopentane-1-carbonylamino)-2,3,4,5-tetrahydro-2-oxo-1H-1-benzazepine-1-acetate, (=compound Ib-13);

Benzyl (3S)-3-(1-phosphonomethyl-cyclopentane-1-carbonylamino)-2,3,4,5-tetrahydro-2-oxo-1H-1-benzazepine-1-acetate, (=compound Ib-14);

Benzyl (3S)-3-(1-diisopropylphosphonomethyl-cyclopentane-1-carbonylamino)-2,3,4,5-tetrahydro-2-oxo-1H-1-benzazepine-1-acetate, (=compound Ib-15);

Ethyl (3S)-3-(1-benzylisopropylphosphonomethyl-cyclopentane-1-carbonylamino)-2,3,4,5-tetrahydro-2-oxo-1H-1-benzazepine-1-acetate, (=compound Ib-16);

tert-Butyl (3S)-3-(1-ethylphosphonomethyl-cyclopentane-1-carbonylamino)-2,3,4,5-tetrahydro-2-oxo-1H-1-benzazepine-1-acetate, (=compound Ib-17);

tert-Butyl (3S)-3-[1-(pivaloyloxymethyl-ethylphosphonomethyl)—cyclopentane-1-carbonylamino]-2,3,4,5-tetrahydro-2-oxo-1H-benzazepine-1-acetate, (=compound Ib-18);

tert-Butyl (3S)-3-(1-phosphonomethyl-cyclopentane-1-carbonylamino)-2,3,4,5-tetrahydro-2-oxo-1H-1-benzazepine-1-acetate, (=compound Ib-19); and physiologically acceptable salts of acids thereof.

Particular preferred examples of compounds of formular Ib are, e.g. compound Ib-2, compound Ib-8, compound Ib-18 or compound Ib-19, most preferably compound Ib-8, and physiologically acceptable salts of acids thereof.

The present invention for the first time provides evidence that, in addition to ECE-1 metalloprotease known previously in the state of the art, the IGS5 type of endothelin converting enzyme also qualifies to be a metalloprotease which is particularly involved in the cleavage of big-ET to ET-1. Therefore, these findings according to the present invention provide new and interesting prospects regarding improved therapeutical concepts for the treatment and/or prophylaxis or inhibition of various diseases influenced and/or implied by IGS5 mediated cleavage of big-ET to ET-1, as the present invention suggests the identification and use of therapeutically active compounds that i.a. specifically inhibit IGS5 type metalloprotease, rather than to look for and to use compounds binding to previously known ECE-1.

It needs to be stressed that the compounds with structure of formula Ia or formula Ib mentioned above were originally selected in the state of the art on the basis of their NEP-inhibitory activity. Thus, the very high potency towards IGS5 activity in vitro surprisingly found for these compounds according to the present invention is paralleled by the capability of said compounds to substantially lessen the pressor effect of big-ET in anaesthetised rats.

In conclusion, the investigations according to the present invention have led to the finding of a novel ECE/NEP-like metalloprotease that efficiently cleaves big-ET and is sensitive not only to the known endothelin converting enzyme inhibitor phosphoramidon, but surprisingly also to a number of defined metalloprotease inhibitors with structures of formula I, preferably with a strutcture of formula Ia or formula Ib. It is therefore conceivable that IGS5 may play a role in the production of ET-1, and that compounds with structures such as formula I, preferably such as formula Ia or formula Ib, may exert their in vivo effects by inhibiting this newly identified IGS5 metalloprotease enzyme.

Therefore, further studies elucidating tissue distribution, physiological function and pathophysiological role of IGS5 in various diseases, preferably in hypertension, renal disease and heart failure, have been initiated in the context of the present invention with compounds showing combined or concurrent NEP/IGS5 inhibitory activity.

In a double-blind placebo-controlled clinical study involving thirteen healthy volunteers compound Ia-2 dose-dependently inhibited the big-ET-induced pressure response and showed a clear dose related increase in ANP levels, indicative of its NEP-inhibitory properties. ET-1 levels did not increase as would have been expected from a selective NEP inhibitor, whereas big-ET levels were increased dose dependently in the compound Ia-2 groups compared to placebo group, indicating that the breakdown of big-ET was also inhibited. To proof the concept of clinical efficacy and safety of compound Ia-2 in man, 6 clinical trials were conducted in healthy volunteers and 2 proof of concept trials were conducted in patients: One randomized, placebo-controlled double-blind trial in patients with hypertension (N=191) and one open, baseline controlled pilot trial in patients with congestive heart failure (N=29). Results showed evidence of neutral endopeptidase (NEP) inhibition by significantly increased and sustained plasma concentrations of ANP and it's second messenger cGMP in both volunteers and patients following oral dosing with compound Ia-2. Furthermore, results in patients with congestive heart failure showed a significant positive correlation between log plasma concentrations of compound Ia-1 (the active metabolite of compound Ia-2) and plasma levels of big-ET following compound Ia-2 administration (p<0.001). In fact plasma levels of big-ET tended to increase following compound Ia-2 administration from baseline (200 mg: 4.9 to 6.5 fmol/ml (+32.6%); 400 mg: 2.3 to 3.5 fmol/ml (+56%)), supporting the concept of activity of orally administered compound Ia-2 to prevent cleavage of big-ET. Most importantly, compound Ia-2 has demonstrated first evidence of significant and clinically relevant anti-hypertensive activity in a recently completed 4-week study (N=191) in patients with WHO grade I-II hypertension. In the Intent-to-treat patient population (ITT), office diastolic blood pressure versus placebo decreased by −6.9 mmHg (last value under treatment; p<0.001) and office systolic blood pressure versus placebo decreased by −9.2 mmHg (last value under treatment; p=0.003) at the highest dose investigated (200 mg bid). This observation is of particular importance as pure NEP inhibitors have been demonstrated to increase ET-1 and consequently rise rather than decrease blood pressure.

Further studies are planned to investigate the dose-response relationship of compound Ia-2 on cardiac hemodynamic parameters in patients with congestive heart failure, and to investigate the time course of anti-hypertensive effects following once daily dosing in patients with hypertension.

IGS5 Metalloproteases in the Context of the Invention

In the context of the present invention reference is made to IGS5 polypeptides (or IGS5 enzymes or IGS5 metalloproteases, e.g. to IGS5PROT, IGS5PROT1 or IGS5PROT2, respectively), in particular to human IGS5 polypeptides (or human IGS5 enzymes). The IGS5 polypeptides may pertain to polypeptides, in particular to human species polypeptides, comprising an amino acid sequence which has at least 70% identity, preferably at least 80% and in particular at least 85% identity, more preferably at least 90% identity, yet more preferably at least 95% identity, most preferably at least 97-99% identity, to one of that selected from the group of SEQ ID NO:2, SEQ ID NO:4 SEQ and SEQ ID NO:6. Such polypeptides include those comprising a IGS5 polypeptide which is identical to one of the amino acid sequences selected from the group of SEQ ID NO:2, SEQ ID NO:4 SEQ and ID NO:6.

Such polypeptides also include those IGS5 polypeptides, in particular human IGS5 polypeptides, having an amino acid sequence of at least 70% identity, preferably at least 80% and in particular at least 85% identity, more preferably at least 90% identity, yet more preferably at least 95% identity, most preferably at least 97-99% identity, to one of the amino acid sequences selected from the group of SEQ ID NO:2, SEQ ID NO:4 SEQ and ID NO:6. Such polypeptides include the IGS5 polypeptides which are identical to one of the amino acid sequences selected from the group of SEQ ID NO:2, of SEQ ID NO:4 and SEQ ID NO:6.

Further polypeptides of the present invention include isolated IGS5 polypeptides comprising the sequence contained in one of SEQ ID NO:2, SEQ ID NO:4 SEQ and ID NO:6, and.

The IGS5 polypeptides in the context of the present invention are members of the neprilysin metalloprotease family, and in particular they are human species polypeptides. They are of interest because several dysfunctions, disorders or diseases have been identified above and in which these newly identified metalloproteases play a critical role in the pathology of the disease.

Thus, according to the present invention it was found that the IGS5 polypeptides may be involved in the metabolism of biologically active peptides, and in particular that these IGS5 polypeptides are metalloprotease type enzymes which may act on a variety of vasoactive peptides. Vasoactive peptides known in the state of the art are e.g. such like atrial natriuretic peptide (ANP), bradykinin, big endothelin (big ET-1), endothelin (ET-1), substance P, and angiotensin-1 Furthermore, it was found that the IGS5 ectodomain, which is a novel human metalloprotease, efficiently hydrolyzes e.g. in vitro a variety of said vasoactive peptides, in particular big-ET-1, bradykinin and substance P.

The IGS5 metalloprotease type enzymes may be inhibited by reference compounds that are used to determine the inhibition properties with regard to enzymes having ECE/NEP-characteristics, e.g. inhibition by compounds such like phosphoramidon. But no inhibition of IGS5 is observed by reference compounds that selectively inhibit NEP, e.g. no inhibition of IGS5 by compounds such as thiorphan, or by reference compounds that selectively inhibit ECE, e.g. no inhibition of IGS5 could be observed for compounds such as SM-19712 (Sumitomo, supra).

Inhibition of IGS5 could be observed at higher concentrations only for reference compounds that inhibit NEP/ECE, e.g. an example is the NEP/ECE inhibitor CGS-35066 (De Lombart et al., supra). The inhibition data of these reference compounds with regard to the inhibition of the IGS5 metalloprotease type enzymes of the present invention are further described in the experimental part below.

The IGS5 polypeptides of the present invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art. Thus, in an example the IGS5 metalloprotease may be generated by methods particularly described in the copending international patent application PCT/EP 00/11532, which is incorporated by reference herein with regard to its entire content, especially with regard to the homology cloning of the human IGS5 gene and to the expression of the corresponding human IGS5 protein.

IGS5 polynucleotides encoding said IGS5 metalloproteases may also be obtained, using standard cloning and screening techniques, from a cDNA library derived from mRNA in cells of human testis tissue, using the expressed sequence tag (EST) analysis (Adams, M. D., et al. Science (1991) 252:1651-1656; Adams, M. D. et al., Nature, (1992) 355:632-634; Adams, M. D., et al., Nature (1995) 377 Supp:3-174). IGS5 polynucleotides can also be obtained from natural sources such as genomic DNA libraries or can be synthesized using well known and commercially available techniques (e.g. F. M. Ausubel et al., 2000, Current Protocols in Molecular Biology).

When IGS5 polynucleotides are used for the recombinant production of the IGS5 polypeptides, the polynucleotide may include the coding sequence for the mature polypeptide, by itself; or the coding sequence for the mature polypeptide in reading frame with other coding sequences, such as those encoding a leader or secretory sequence, a pre-, or pro- or prepro-protein sequence, or other fusion peptide portions. For example, a marker sequence which facilitates purification of the fused polypeptide can be encoded. For example, the marker sequence may preferably be a hexa-histidine peptide, as provided in the pQE vector (Qiagen, Inc.) and described in Gentz et al., Proc Natl Acad Sci USA (1989) 86:821-824, or is an HA tag. The polynucleotide may also contain non-coding 5′ and 3′ sequences, such as transcribed, non-translated sequences, splicing and polyadenylation signals, ribosome binding sites and sequences that stabilize mRNA. IGS5 polynucleotides which are identical or sufficiently identical to a nucleotide sequence contained in one of SEQ ID NO:1, SEQ ID NO:3 or SEQ ID NO:5, may be used as hybridization probes for cDNA and genomic DNA or as primers for a nucleic acid amplification (PCR) reaction, to isolate full-length cDNAs and genomic clones encoding IGS5 polypeptides and to isolate cDNA and genomic clones of other genes (including genes encoding paralogs from human sources and orthologs and paralogs from species other than human) that have a high sequence similarity to one of SEQ ID NO:1, SEQ ID NO:3 or SEQ ID NO:5. Typically these nucleotide sequences are at least 70% identical, preferably at least 80% and in particular at least 85% identical, more preferably at least 90% identical, most preferably at least 95% identical to that of the referent. The probes or primers will generally comprise at least 15 nucleotides, preferably, at least 30 nucleotides and may have at least 50 nucleotides. Particularly preferred probes will have between 30 and 50 nucleotides. Particularly preferred primers will have between 20 and 25 nucleotides.

An IGS5 polynucleotide encoding an IGS5 polypeptide, in particular a human IGS5 polypeptide, may be obtained by a process which comprises the steps of screening an appropriate library under stringent hybridization conditions with a labeled probe having the sequence of one of SEQ ID NO:1, SEQ ID NO:3 or SEQ ID NO:5, or a fragment thereof; and isolating full-length cDNA and genomic clones containing said polynucleotide sequence. Such hybridization techniques are well known to the skilled artisan. Preferred stringent hybridization conditions include overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate (w/v), and 20 μg/ml denatured, sheared salmon sperm DNA; followed by washing the filters in 0.1×SSC at about 65° C. Thus, IGS5 polynucleotides may be obtained by screening an appropriate library under stringent hybridization conditions with a labeled probe having the sequence of one of SEQ ID NO:1, SEQ ID NO:3 or SEQ ID NO:5, or a fragment thereof.

The skilled artisan will appreciate that, in many cases, an isolated cDNA sequence will be incomplete, in that the region coding for the polypeptide is cut short at the 5′ end of the cDNA. This is a consequence of reverse transcriptase, an enzyme with inherently low “processivity” (a measure of the ability of the enzyme to remain attached to the template during the polymerisation reaction), failing to complete a DNA copy of the mRNA template during 1st strand cDNA synthesis. There are several methods available and well known to those skilled in the art to obtain full-length cDNAs, or extend short cDNAs, for example those based on the method of Rapid Amplification of cDNA ends (RACE) (see, for example, Frohman et al., PNAS USA 85, 8998-9002, 1988). Recent modifications of the technique, exemplified by the Marathon™ technology (Clontech Laboratories Inc.) for example, have significantly simplified the search for longer cDNAs. In the Marathon™ technology, cDNAs have been prepared from mRNA extracted from a chosen tissue and an “adaptor” sequence ligated onto each end. Nucleic acid amplification (PCR) is then carried out to amplify the “missing” 5′ end of the cDNA using a combination of gene specific and adaptor specific oligonucleotide primers. The PCR reaction is then repeated using “nested” primers, that is, primers designed to anneal within the amplified product (typically an adaptor specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Recombinant IGS5 polypeptides may be prepared by processes well known in the art from genetically engineered host cells comprising expression systems which comprise an IGS5 polynucleotide or polynucleotides. Host cells which are genetically engineered with such expression systems may be used for the production of IGS5 polypeptides by recombinant techniques. Cell-free translation systems can also be employed to produce such IGS5 proteins using RNAs derived from IGS5 DNA constructs. Introduction of IGS5 polynucleotides into host cells can be effected by methods described in many standard laboratory manuals, such as Davis et al., Basic Methods in Molecular Biology (1986) and Sambrook et al., Molecular Cloning: A Laboratory Manual, 2nd Ed., Cold Spring Harbor Laboratory Press, Cold Spring Harbor, N.Y. (1989). Such methods include, for instance, calcium phosphate transfection, DEAE-dextran mediated transfection, transvection, microinjection, cationic lipid-mediated transfection, electroporation, transduction, scrape loading, ballistic introduction or infection. Representative examples of appropriate hosts include bacterial cells, such as Streptococci, Staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector which is able to maintain, propagate or express a polynucleotide to produce a polypeptide in a host may be used. The appropriate nucleotide sequence may be inserted into an expression system by any of a variety of well-known and routine techniques, such as, for example, those set forth in Sambrook et al., Molecular Cloning, A Laboratory Manual (supra). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals, i.e. derived from a different species.

If an polypeptide is to be expressed for use in screening assays, Generally it is possible that the IGS5 polypeptide is produced at the surface of the cell or alternatively in a soluble protein form. If the IGS5 polypeptide is secreted into the medium, the medium can be recovered in order to recover and purify the IGS5 polypeptide. If produced intracellularly, the cells must first be lysed before the IGS5 polypeptide is recovered. If the IGS5 polypeptide is bound at the surface of the cell (membrane bound polypeptide), usually membrane fractions are prepared in order to accumulate the membrane bound IGS5 polypeptide. IGS5 polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during intracellular synthesis, isolation and or purification.

Isolated IGS5 polynucleotides, in particular isolated human IGS5 polynucleotides, that may be used to generate an IGS5 polypeptide usually comprise a nucleotide sequence that has at least 70% identity, preferably at least 80% and in particular at least 85% identity, more preferably at least 90% identity, yet more preferably at least 95% identity, to a nucleotide sequence encoding one of the polypeptides selected from the group of SEQ ID NO:2, SEQ ID NO:4 and SEQ ID NO:6, over the entire coding region. In this regard, polynucleotides which have at least 97% identity are highly preferred, whilst those with at least 98-99% identity are more highly preferred, and those with at least 99%, in particular 99.9%, identity are most highly preferred. For example, such isolated, in particular human, IGS5 polynucleotides that may be used to generate IGS5 polypeptides include nucleotide sequences which have at least 70% identity, preferably at least 80% and in particular at least 85% identity, more preferably at least 90% identity, yet more preferably at least 95% identity, over the entire length to one of the nucleotide sequences selected from the group of SEQ ID NO:1, SEQ ID NO:3 and SEQ ID NO: 5. In this regard, IGS5 polynucleotides which comprise or have a nucleotide sequence of at least 97% identity to one of the nucleotide sequences selected from the group of SEQ ID NO:1, SEQ ID NO:3 and SEQ ID NO:5 are highly preferred, whilst those with at least 98-99% identity, are more highly preferred, and those with at least 99%, in particular 99.9%, identity are most highly preferred. The IGS5 polynucleotide sequence of SEQ ID NO:1 (designated “IGS5DNA”) is indicated in Table 1 representing a cDNA sequence from human origin (Homo sapiens) with a length of 2076 nucleotides and comprises a polypeptide encoding sequence (from nucleotide no. 1 to no. 2073) encoding a polypeptide of 691 amino acids, the polypeptide of SEQ ID NO:2 (designated “IGS5PROT”) which is indicated in Table 2. The nucleotide sequence of SEQ ID NO:3 (designated “IGS5DNA1”) is indicated in Table 3 representing a cDNA sequence from human origin (Homo sapiens) with a length of 2340 nucleotides (including the stop codon tag) and comprises a polypeptide encoding sequence (from nucleotide no. 1 to no. 2337) encoding a polypeptide of 779 amino acids, the polypeptide of SEQ ID NO:4 (designated “IGS5PROT1”) which is indicated in Table 4. The nucleotide sequence of SEQ ID NO:5 (designated “IGS5DNA2”) is indicated in Table 5 representing a cDNA sequence from human origin (Homo sapiens) with a length of 2262 nucleotides (including the stop codon tag) and comprises a polypeptide encoding sequence (from nucleotide no. 1 to no. 2259) encoding a polypeptide of 753 amino acids, the polypeptide of SEQ ID NO:6 (designated “IGS5PROT2”) which is indicated in Table 6.

Compounds with Combined or Concurrent Selective NEP/IGS5-Inhibitory Activity as New Therapeutical Concept

The findings of the present invention have shown that IGS5 metalloproteases, e.g. also in combination with at least one other metalloprotease such as in particular NEP, and optionally in addition ECE and/or ACE, are responsible for one or more biological functions related to the diseases mentioned herein before. Thus, in its broadest aspect the invention generally provides new therapeutic concepts for the treatment of said diseases, as stated already above, by suggesting for the first time to use compounds with combined or concurrent inhibitory activity on neutral endopeptidase (NEP) and on the metalloprotease IGS5, or a pharmaceutically acceptable salt or solvate or biolabile ester thereof, for the manufacture of a medicament (pharmaceutical composition) for treating a larger mammal, preferably a human, suffering from or being susceptible to a condition which can be alleviated or prevented by combined or concurrent inhibition of NEP and IGS5.

Such compounds useful according to the invention in that they concurrently inhibit the function of the IGS5 metalloprotease and of NEP may be identified by screening methods using IGS5 metalloprotease, and optionally NEP, in an appropriate enzyme inhibition assay format. Such enzyme inhibition assay formats are described in more detail in the experimental section below. For identification of compounds with combined or concurrent selective NEP/IGS5-inhibitory activity candidate compounds may be testet separately in both, an NEP-inhibition assay and IGS5-inhibition assay. NEP/IGS5-inhibitory compounds may be identified from a variety of sources, for example, cells, cell-free preparations, chemical libraries, and natural product mixtures.

The screening method may simply measure the influence of a candidate compound on the activity of the polypeptide excreted into a culture medium, or on cells or membranes bearing the polypeptide. Alternatively, the screening method may involve competition with a competitor. Further, these screening methods may test whether the candidate compound results in a signal generated by activation or inhibition of the polypeptide, using detection systems appropriate to the activity of the polypeptide excreted into a culture medium or to the cells or membranes bearing the polypeptide. Inhibition of polypeptide activity is generally assayed in the presence of a known substrate and the effect of the candidate compound is observed by altered activity, e.g. by testing whether the candidate compound results in inhibition of the polypeptide. For example, the screening methods may simply comprise the steps of mixing a candidate compound with a solution containing a polypeptide of interest in the context of the present invention, and a suitable substrate to form a mixture, measuring the polypeptide activity in the mixture, and comparing the polypeptide activity of the mixture to a standard without candidate compound.

The present invention also enables the person skilled in the art to identify compounds, e.g. candidate compounds, by means of screening methods involving the findings of the present invention, said compounds may reveal as prospective drug candidates in particular with respect to dysfunctions, disorders or diseases that are referenced already above. It will be readily appreciated by the skilled artisan that an IGS5 metalloprotease may also be used in a method for the structure-based design of IGS5 inhibitory compounds, by:

(a) determining or using in the first instance the three-dimensional structure of the IGS5 metalloprotease;

(b) deducing the three-dimensional structure for the likely reactive or binding site(s) of an IGS5 inhibitor;

(c) synthesizing candidate compounds that are predicted to bind to or react with the deduced binding or reactive site; and

(d) testing whether the candidate compounds are indeed IGS5 inhibitors.

It will be further appreciated that this will normally be an iterative process.

Today, medicinal chemists are well aware of modern strategies for planning and performing organic synthesis in order to generate new substances or compounds that are worth to be investigated for potential physiological or pharmacological properties, and which compounds therefore promise to prove as prospective new drug candidates for the treatment and/or prophylaxis or inhibition of specific dysfunctions, disorders or diseases. Furthemore, today it is common to provide compound libraries by means of combinatorial chemistry, e.g. in particular of general and of “directed” chemical or compound libraries, in which the structure and the variations of pharmacophore groups and the residues or substituents are known to the concerned artisan. If chemical libraries or compound libraries with still unknown structure of the compounds are investigated in screening assays, potential prospective compounds, e.g. candidate compounds, nevertheless, may easily be analysed in their structure and chemical properties by today's well-established analytical means such as e.g. mass spectroscopy, nuclear magnetic resonance, infrared spectra, melting points, optical rotation if chiral compounds are involved, and elemental analysis.

Thus the invention also pertains to a process for preparing a candidate compound with a defined chemical structure capable of inhibiting the IGS5 polypeptide, said process is comprising the manufacture of a compound or of a pharmaceutically acceptable salt or biolabile ester thereof by means of chemical synthesis, provided that the activity of the compound to inhibit the IGS5 polypeptide is identifiable by a screening method, e.g. such as described in the experimental section of the present invention.

For details of e.g. chemical organic synthesis, and e.g. chemical, analytical and physical methods see the Handbook “Houben-Weyl” (Houben-Weyl, “Methoden der organischen Chemie”, Georg Thieme Verlag, Stuttgart, N.Y.) in its most recent version.

One embodiment of the present invention pertains to the use of a compound of formula I as given supra or a pharmaceutically acceptable salt or solvate or biolabile ester thereof, for the manufacture of a medicament (pharmaceutical composition) for treating a larger mammal, preferably a human, suffering from or being susceptible to a condition which can be improved or prevented by combined or concurrent inhibition of

a) neutral endopeptidase (NEP) and

b) of the metalloprotease IGS5 which is a polypeptide comprising an amino acid sequence which has at least 70% identity 6 over the entire length to one of the amino acid sequences selected from the group of SEQ ID NO:2, SEQ ID NO:4 and SEQ ID NO: 6.

A preferred embodiment of the present invention pertains to the use of a compounds according to the invention, in particular compunds with formula I, having combined or concurrent inhibitory activity on

a) neutral endopeptidase (NEP) and

b) on the metalloprotease IGS5 which is a polypeptide comprising an amino acid sequence which has at least 70% identity over the entire length to one of the amino acid sequences selected from the group of SEQ ID NO:2, SEQ ID NO:4 and SEQ ID NO:6;

or a pharmaceutically acceptable salt or solvate or biolabile ester thereof, for the manufacture of a medicament (pharmaceutical composition) for treatment and/or prophylaxis or inhibition of hypertension, including secondary forms of hypertension such as renal or pulmonary hypertension, heart failure, angina pectoris, arrhythmias, myocardial infarction, cardiac hypertrophy, cerebral ischemia, peripheral vascular disease, subarachnoidal hemorrhage, chronic obstructive pulmonary disease (COPD), asthma, renal disease, atherosclerosis, and pain in colorectal cancer or prostate cancer, in larger mammals, preferably in humans.

Compounds of formula I can be prepared according to the disclosure in U.S. Pat. No. 5,677,297 which is suitable for compounds of formula Ia and according to U.S. Pat. No. 5,952,327 which is suitable for compounds of formula Ib.

Protein-Ligand Complexes in Drug Design and Lead Structure Optimization

In another aspect the invention relates to a protein-ligand-complex comprising an IGS5 polypeptide of at least 70% identity to one of the polypeptides of SEQ ID NO:2, SEQ ID NO:4 and SEQ ID NO:5 and an IGS5-binding compound, preferably a compound with IGS5-inhibitory activity of at least that of or being comparable to that of compounds of formula I. Such protein-ligand-complexes are particularly useful in drug design methods, lead structure finding, lead structure optimization and modulation methods. The methods are well known in the state of the art. For exemplary reference see literaure concerning e.g. combinatorial synthesis and multidimensional NMR-spectroscopy and its contribution to the understanding of protein-ligand-interactions (Kessler, Angew. Chem. 1997, 109, 857-859; James K. Chen et al., Angew. Chem. 107 (1995), S. 1041-1058). Furthermore see Fesik (Journal of Medicinal Chemistry, 34 (1991), S. 2937-2945) who describes NMR studies of molecular complexes as a tool in drug design; and. Fesik et al. (Biochemical Pharmacology 40 (1990), S. 161-167) who describe NMR methods for determining the structures of enzyme/inhibitor complexes as an aid in drug design. A very recent report of Ross et al. (Journal of Biomolecular NMR, 16: 139-146 (2000)) describes the automation of NMR measurements and data evaluation for systematically screening interactions of small molecules with target proteins, e.g. receptors.

Thus, the invention also pertains to the use of a protein-ligand-complex comprising an IGS5 polypeptide of at least 70% identity to one of the polypeptides of SEQ ID NO:2, SEQ ID NO:4 and SEQ ID NO:6 and an IGS5-binding compound for the design and modulation or optimization of lead structures with IGS5-binding and IGS5-inhibitory activity.

Combination Therapy (NEP/IGS5-Inhibitory Compounds and ECE/ACE-Inhibitory Compounds)

As already shortly addressed supra, it may be beneficial to additionally combine compounds showing combined or concurrent NEP/IGS5 inhibitory activity according to the invention, e.g. compounds of formula I, preferably compounds of formula Ia or Ib, with other individual and/or combined metalloprotease inhibitors than combined NEP/IGS5 inhibitors. Such other metalloprotease inhibitors that may be used in combination with said compounds with combined NEP/IGS5 inhibitory activity are for example ACE inhibitors such as captopril, enalapril, lisinopril, fosinopril, perindopril, quinapril, ramipril; furthermore, selective ECE inhibitors such as compound SM-19712 (Sumitomo, supra); selective NEP inhibitors such as thiorphan; dual NEP/ECE inhibitors such as compound CGS-35066 (De Lombart et al., J. Med. Chem. 2000, Feb. 10; 43(3):488-504); or mixed inhibitors of these metalloproteases such as omapatrilat or sampatrilat. By this type of combination treatment and/or prophylaxis or inhibition the therapeutic value of the compounds with combined or concurrent NEP/IGS5 inhibitory activity still may be further increased, in particular with regard to the diseases and/or conditions mentioned above. Therefore in a further aspect the invention particularly also pertains to a combination therapy and/or combination prophylaxis or inhibition. By this type of combination treatment and/or prophylaxis or inhibition the therapeutic value of said compounds with combined or concurrent NEP/IGS5 inhibitory activity, in particular of compounds with formula I, preferably of compounds with formula Ia of Ib, still may be further increased, in particular with regard to the diseases and/or conditions mentioned above.

Thus, in this respect the invention pertains to the use of a first compound showing combined or concurrent NEP/IGS5 inhibitory activity or a pharmaceutically acceptable salt or solvate or biolabile ester thereof, as these are described above with regard to the present invention, in combination with at least one additional compound selected from the group of other individual and/or combined metalloprotease inhibitors than the combined NEP/IGS5 inhibitors, said additional compound preferably being selected from the group of ACE inhibitors, selective ECE inhibitors, selective NEP inhibitors, dual NEP/ECE inhibitors, and mixed inhibitors of these metalloproteases, for the manufacture of a medicament (pharmaceutical composition) for combination treatment and/or combination prophylaxis or inhibition of any of the diseases or conditions as referenced above in the context of the present invention. Particularly this use according to the present invention of said first compound in combination with at least one of said additional compounds, is characterized in that the first compound compound has a structure of formula I, preferably a structure of formula Ia or of formula Ib, as these formulas are referenced above in the context of the present invention. Preferably, the use of said first compound in combination with at least one of said additional compounds, is further characterized in that the combination is co-effective, preferably synergistically effective.

Furthermore, the invention in this respect pertains to pharmaceutical composition (medicament), comprising co-effective, preferably synergistically effective, amounts of: a first compound with combined or concurrent NEP/IGS5 inhibitory activity or a pharmaceutically acceptable salt or solvate or biolabile ester thereof, as these are described above with regard to the present invention; and of at least one additional compound selected from the group of other individual and/or combined metalloprotease inhibitors than the combined NEP/IGS5 inhibitors, said additional compound preferably being selected from the group of ACE inhibitors, selective ECE inhibitors, selective NEP inhibitors, dual NEP/ECE inhibitors, and mixed inhibitors of these metalloproteases, for combination treatment and/or combination prophylaxis or inhibition of any of the diseases or conditions as referenced above in the context of the present invention. In particular the pharmaceutical composition according to the present invention may comprise co-effective, preferably synergistically effective, amounts of said first compound and of at least one of said additional compounds, being further characterized in that the first compound compound has a structure of formula I, preferably a structure of formula Ia or of formula Ib, as these formulas are referenced above in the context of the present invention.

It is self explaining to the skilled person that combination therapy and/or combination prophylaxis or inhibition according to the present invention may be achieved by administering to a patient in need of such a therapy and/or such prophylaxis or inhibition the first compound with combined or concurrent NEP/IGS5 inhibitory activity or a pharmaceutically acceptable salt or solvate or biolabile ester thereof and the additional compound selected from the group of other individual and/or combined metalloprotease inhibitors than the combined NEP/IGS5 inhibitors, in a simultaneous manner, either by administering a single pharmaceutical combination preparation or by separate pharmaceutical preparation for the first and the second compound, in a separate manner, e.g. under a given dosage regimen or scheme which may be either continous or sequential, or in a graded manner, whatever seems suitable with regard to the patients disease or condition to be alleviated and/or prevented.

Formulation and Administration

The foregoing findings according to the invention show that combined or concurrent selective NEP/IGS5-inhibitory compounds, optionally in combination with separate ACE- and/or ECE-inhibitory compounds, or their respective pharmaceutically acceptable salts or solvates or biolabile esters thereof exert a beneficial therapeutic activity. Therefore these compounds, optionally in combination with separate ACE- and/or ECE-inhibitors, are suitable as medicaments for the treatment and/or prophylaxis or inhibition of hypertension, including secondary forms of hypertension such as renal or pulmonary hypertension, heart failure, angina pectoris, arrhythmias, myocardial infarction, cardiac hypertrophy, cerebral ischemia, peripheral vascular disease, subarachnoidal hemorrhage, chronic obstructive pulmonary disease (COPD), asthma, renal disease, atherosclerosis, and pain in colorectal cancer or prostate cancer, in larger mammals, especially in humans. NEP/IGS5-inhibitory compounds, optionally in combination with separate ACE- and/or ECE-inhibitory compounds, may be given by all known administration routes.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition compounds can be formulated in an enteric or an encapsulated formulation, oral administration may also be possible. Administration of these compounds may also be topical and/or localized, in the form of salves, pastes, gels, and the like.

For administration according to the invention the therapeutically active quantities of the NEP/IGS5-inhibitory compounds that alleviate and/or prevent the diseases or conditions mentioned supra in the context of the invention can be contained together with customary pharmaceutical excipients and/or additives in solid or liquid pharmaceutical formulations.

Examples of solid dosage forms are such as solid, semi-solid, lyophilized powder, tablets, coated tablets, pills, capsules, powders, granules or suppositories, also in form of sustained release formulations. These solid dosage forms can contain standard pharmaceutical inorganic and/or organic excipients. Such excipients include pharmaceutical grades of mannitol, lactose, starch, magnesium stearate, sodium saccharine, talcum, cellulose, glucose, gelatin, sucrose, magnesium carbonate, and the like in addition to customary pharmaceutical additives such as fillers, lubricants or tablet disintegrants. Liquid preparations such as solutions, suspensions or emulsions of the active ingredients can contain the usual diluents such as water, oil and/or suspending aids such as polyethylene glycols and such like. Further additives such as preservatives, flavoring agents and such like may also be added.

The active ingredients can be mixed and formulated with the pharmaceutical excipients and/or additives in a known manner. For the manufacture of solid dosage forms, for example, the active ingredients may be mixed with the excipients and/or additives and granulated in a wet or dry process. Granules or powder can be filled directly into capsules or compressed into tablet cores. If desired, these can be coated in the known manner.

Liquid preparations can be prepared by dissolving or dispersing the compounds and optional pharmaceutical adjuvants, in a carrier, such as, for example, aqueous saline, aqueous dextrose, glycerol, or ethanol, to form a solution or suspension.

The doses to be administered may differ between individuals and naturally vary depending on the type of condition to be treated and the route of administration. For example, locally applicable formulations injectable formulations, generally contain substantially less amount of active substance than systemically applicable formulations. Thus, the dosage range required depends on the judgment of the attending practitioner, in particular in view of the choice of compounds, the route of administration, the nature of the formulation, and the nature of the subject's condition. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Tables with IGS5 DNA and IGS5 Protein Sequences

TABLE 1
IGS5-DNA (“IGS5DNA”) of SEQ ID NO:1

5′-
TGCACCACCCCTGGCTGCGTGATAGCAGCTGCCAGGATCCTCCAGAACATGGACCCGACC
ACGGAACCGTGTGACGACTTCTACCAGTTTGCATGCGGAGGCTGGCTGCGGCGCCACGTG
ATCCCTGAGACCAACTCAAGATACAGCATCTTTGACGTCCTCCGCGACGAGCTGGAGGTC
ATCCTCAAAGCGGTGCTGGAGAATTCGACTGCCAAGGACCGGCCGGCTGTGGAGAAGGCC
AGGACGCTGTACCGCTCCTGCATGAACCAGAGTGTGATAGAGAAGCGAGGCTCTCAGCCC
CTGCTGGACATCTTGGAGGTGGTGGGAGGCTGGCCGGTGGCGATGGACAGGTGGAACGAG
ACCGTAGGACTCGAGTGGGAGCTGGAGCGGCAGCTGGCGCTGATGAACTCACAGTTCAAC
AGGCGCGTCCTCATCGACCTCTTCATCTGGAACGACGACCAGAACTCCAGCCGGCACATC
ATCTACATAGACCAGCCCACCTTGGGCATGCCCTCCCGAGAGTACTACTTCAACGGCGGC
AGCAACCGGAAGGTGCGGGAAGCCTACCTGCAGTTCATGGTGTCAGTGGCCACGTTGCTG
CGGGAGGATGCAAACCTGCCCAGGGACAGCTGCCTGGTGCAGGAGGACATGATGCAGGTG
CTGGAGCTGGAGACACAGCTGGCCAAGGCCACGGTACCCCAGGAGGAGAGACACGACGTC
ATCGCCTTGTACCACCGGATGGGACTGGAGGAGCTGCAAAGCCAGTTTGGCCTGAAGGGA
TTTAACTGGACTCTGTTCATACAAACTGTGCTATCCTCTGTCAAAATCAAGCTGCTGCCA
GATGAGGAAGTGGTGGTCTATGGCATCCCCTACCTGCAGAACCTTGAAAACATCATCGAC
ACCTACTCAGCCAGGACCATACAGAACTACCTGGTCTGGCGCCTGGTGCTGGACCGCATT
GGTAGCCTAAGCCAGAGATTCAAGGACACACGAGTGAACTACCGCAAGGCGCTGTTTGGC
ACAATGGTGGAGGAGGTGCGCTGGCGTGAATGTGTGGGCTACGTCAACAGCAACATGGAG
AACGCCGTGGGCTCCCTCTACGTCAGGGAGGCGTTCCCTGGAGACAGCAAGAGCATGGTC
AGAGAACTCATTGACAAGGTGCGGACAGTGTTTGTGGAGACGCTGGACGAGCTGGGCTGG
ATGGACGAGGAGTCCAAGAAGAAGGCGCAGGAGAAGGCCATGAGCATCCGGGAGCAGATC
GGGCACCCTGACTACATCCTGGAGGAGATGAACAGGCGCCTGGACGAGGAGTACTCCAAT
CTGAACTTCTCAGAGGACCTGTACTTTGAGAACAGTCTGCAGAACCTCAAGGTGGGCGCC
CAGCGGAGCCTCAGGAAGCTTCGGGAAAAGGTGGACCCAAATCTCTGGATCATCGGGGCG
GCGGTGGTCAATGCGTTCTACTCCCCAAACCGAAACCAGATTGTATTCCCTGCCGGGATC
CTCCAGCCCCCCTTCTTCAGCAAGGAGCAGCCACAGGCCTTGAACTTTGGAGGCATTGGG
ATGGTGATCGGGCACGAGATCACGCACGGCTTTGACGACAATGGCCGGAACTTCGACAAG
AATGGCAACATGATGGATTGGTGGAGTAACTTCTCCACCCAGCACTTCCGGGAGCAGTCA
GAGTGCATGATCTACCAGTACGGCAACTACTCCTGGGACCTGGCAGACGAACAGAACGTG
AACGGATTCAACACCCTTGGGGAAAACATTGCTGACAACGGAGGGGTGCGGCAAGCCTAT
AAGGCCTACCTCAAGTGGATGGCAGAGGGTGGCAAGGACCAGCAGCTGCCCGGCCTGGAT
CTCACCCATGAGCAGCTCTTCTTCATCAACTACGCCCAGGTGTGGTGCGGGTCCTACCGG
CCCGAGTTCGCCATCCAATCCATCAAGACAGACGTCCACAGTCCCCTGAAGTACAGGGTA
CTGGGGTCGCTGCAGAACCTGGCCGCCTTCGCAGACACGTTCCACTGTGCCCGGGGCACC
CCCATGCACCCCAAGGAGCGATGCCGCGTGTGGTAG - 3′

TABLE 2
IGS5-protein (“IGS5PROT”) of SEQ ID NO:2

CTTPGCVIAAARILQNMDPTTEPCDDFYQFACGGWLRRHVIPETNSRYSIFDVLRDELEV
ILKAVLENSTAKDRPAVEKARTLYRSCMNQSVIEKRGSQPLLDILEVVGGWPVAMDRWNE
TVGLEWELERQLALMNSQFNRRVLIDLFIWNDDQNSSRHIIYIDQPTLGMPSREYYFNGG
SNRKVREAYLQFMVSVATLLREDANLPRDSCLVQEDMMQVLELETQLAKATVPQEERHDV
IALYHRMGLEELQSQFGLKGFNWTLFIQTVLSSVKIKLLPDEEVVVYGIPYLQNLENIID
TYSARTIQNYLVWRLVLDRIGSLSQRFKDTRVNYRKALFGTMVEEVRWRECVGYVNSNME
NAVGSLYVREAFPGDSKSMVRELIDKVRTVFVETLDELGWMDEESKKKAQEKAMSIREQI
GHPDYILEEMNRRLDEEYSNLNFSEDLYFENSLQNLKVGAQRSLRKLREKVDPNLWIIGA
AVVNAFYSPNRNQIVFPAGILQPPFFSKEQPQALNFGGIGMVIGHEITHGFDDNGRNFDK
NGNMMDWWSNFSTQHFREQSECMIYQYGNYSWDLADEQNVNGFNTLGENIADNGGVRQAY
KAYLKWMAEGGKDQQLPGLDLTHEQLFFINYAQVWCGSYRPEFAIQSIKTDVHSPLKYRV
LGSLQNLAAFADTFHCARGTPMHPKERCRVW

TABLE 3
IGS5-DNA-1 (“IGS5DNA1”) of SEQ ID NO:3

5′-
ATGGGGAAGTCCGAAGGCCCCGTGGGGATGGTGGAGAGCGCTGGCCGTGCAGGGCAGAAG
CGCCCGGGGTTCCTGGAGGGGGGGCTGCTGCTGCTGCTGCTGCTGGTGACCGCTGCCCTG
GTGGCCTTGGGTGTCCTCTACGCCGACCGCAGAGGGAAGCAGCTGCCACGCCTTGCTAGC
CGGCTGTGCTTCTTACAGGAGGAGAGGACCTTTGTAAAACGAAAACCCCGAGGGATCCCA
GAGGCCCAAGAGGTGAGCGAGGTCTGCACCACCCCTGGCTGCGTGATAGCAGCTGCCAGG
ATCCTCCAGAACATGGACCCGACCACGGAACCGTGTGACGACTTCTACCAGTTTGCATGC
GGAGGCTGGCTGCGGCGCCACGTGATCCCTGAGACCAACTCAAGATACAGCATCTTTGAC
GTCCTCCGCGACGAGCTGGAGGTCATCCTCAAAGCGGTGCTGGAGAATTCGACTGCCAAG
GACCGGCCGGCTGTGGAGAAGGCCAGGACGCTGTACCGCTCCTGCATGAACCAGAGTGTG
ATAGAGAAGCGAGGCTCTCAGCCCCTGCTGGACATCTTGGAGGTGGTGGGAGGCTGGCCG
GTGGCGATGGACAGGTGGAACGAGACCGTAGGACTCGAGTGGGAGCTGGAGCGGCAGCTG
GCGCTGATGAACTCACAGTTCAACAGGCGCGTCCTCATCGACCTCTTCATCTGGAACGAC
GACCAGAACTCCAGCCGGCACATCATCTACATAGACCAGCCCACCTTGGGCATGCCCTCC
CGAGAGTACTACTTCAACGGCGGCAGCAACCGGAAGGTGCGGGAAGCCTACCTGCAGTTC
ATGGTGTCAGTGGCCACGTTGCTGCGGGAGGATGCAAACCTGCCCAGGGACAGCTGCCTG
GTGCAGGAGGACATGATGCAGGTGCTGGAGCTGGAGACACAGCTGGCCAAGGCCACGGTA
CCCCAGGAGGAGAGACACGACGTCATCGCCTTGTACCACCGGATGGGACTGGAGGAGCTG
CAAAGCCAGTTTGGCCTGAAGGGATTTAACTGGACTCTGTTCATACAAACTGTGCTATCC
TCTGTCAAAATCAAGCTGCTGCCAGATGAGGAAGTGGTGGTCTATGGCATCCCCTACCTG
CAGAACCTTGAAAACATCATCGACACCTACTCAGCCAGGACCATACAGAACTACCTGGTC
TGGCGCCTGGTGCTGGACCGCATTGGTAGCCTAAGCCAGAGATTCAAGGACACACGAGTG
AACTACCGCAAGGCGCTGTTTGGCACAATGGTGGAGGAGGTGCGCTGGCGTGAATGTGTG
GGCTACGTCAACAGCAACATGGAGAACGCCGTGGGCTCCCTCTACGTCAGGGAGGCGTTC
CCTGGAGACAGCAAGAGCATGGTCAGAGAACTCATTGACAAGGTGCGGACAGTGTTTGTG
GAGACGCTGGACGAGCTGGGCTGGATGGACGAGGAGTCCAAGAAGAAGGCGCAGGAGAAG
GCCATGAGCATCCGGGAGCAGATCGGGCACCCTGACTACATCCTGGAGGAGATGAACAGG
CGCCTGGACGAGGAGTACTCCAATCTGAACTTCTCAGAGGACCTGTACTTTGAGAACAGT
CTGCAGAACCTCAAGGTGGGCGCCCAGCGGAGCCTCAGGAAGCTTCGGGAAAAGGTGGAC
CCAAATCTCTGGATCATCGGGGCGGCGGTGGTCAATGCGTTCTACTCCCCAAACCGAAAC
CAGATTGTATTCCCTGCCGGGATCCTCCAGCCCCCCTTCTTCAGCAAGGAGCAGCCACAG
GCCTTGAACTTTGGAGGCATTGGGATGGTGATCGGGCACGAGATCACGCACGGCTTTGAC
GACAATGGCCGGAACTTCGACAAGAATGGCAACATGATGGATTGGTGGAGTAACTTCTCC
ACCCAGCACTTCCGGGAGCAGTCAGAGTGCATGATCTACCAGTACGGCAACTACTCCTGG
GACCTGGCAGACGAACAGAACGTGAACGGATTCAACACCCTTGGGGAAAACATTGCTGAC
AACGGAGGGGTGCGGCAAGCCTATAAGGCCTACCTCAAGTGGATGGCAGAGGGTGGCAAG
GACCAGCAGCTGCCCGGCCTGGATCTCACCCATGAGCAGCTCTTCTTCATCAACTACGCC
CAGGTGTGGTGCGGGTCCTACCGGCCCGAGTTCGCCATCCAATCCATCAAGACAGACGTC
CACAGTCCCCTGAAGTACAGGGTACTGGGGTCGCTGCAGAACCTGGCCGCCTTCGCAGAC
ACGTTCCACTGTGCCCGGGGCACCCCCATGCACCCCAAGGAGCGATGCCGCGTGTGGTAG
- 3′

TABLE 4
IGS5-protein-1 (“IGS5PROT1”) of SEQ ID NO:4

MGKSEGPVGMVESAGRAGQKRPGFLEGGLLLLLLLVTAALVALGVLYADRRGKQLPRLAS
RLCFLQEERTFVKRKPRGIPEAQEVSEVCTTPGCVIAAARILQNMDPTTEPCDDFYQFAC
GGWLRRHVIPETNSRYSIFDVLRDELEVILKAVLENSTAKDRPAVEKARTLYRSCMNQSV
IEKRGSQPLLDILEVVGGWPVAMDRWNETVGLEWELERQLALMNSQFNRRVLIDLFIWND
DQNSSRHIIYIDQPTLGMPSREYYFNGGSNRKVREAYLQFMVSVATLLREDANLPRDSCL
VQEDMMQVLELETQLAKATVPQEERHDVIALYHRMGLEELQSQFGLKGFNWTLFIQTVLS
SVKIKLLPDEEVVVYGIPYLQNLENIIDTYSARTIQNYLVWRLVLDRIGSLSQRFKDTRV
NYRKALFGTMVEEVRWRECVGYVNSNMENAVGSLYVREAFPGDSKSMVRELIDKVRTVFV
ETLDELGWMDEESKKKAQEKAMSIREQIGHPDYILEEMNRRLDEEYSNLNFSEDLYFENS
LQNLKVGAQRSLRKLREKVDPNLWIIGAAVVNAFYSPNRNQIVFPAGILQPPFFSKEQPQ
ALNFGGIGMVIGHEITHGFDDNGRNFDKNGNMMDWWSNFSTQHFREQSECMIYQYGNYSW
DLADEQNVNGFNTLGENIADNGGVRQAYKAYLKWMAEGGKDQQLPGLDLTHEQLFFINYA
QVWCGSYRPEFAIQSIKTDVHSPLKYRVLGSLQNLAAFADTFHCARGTPMHPKERCRVW

TABLE 5
IGS5-DNA-2 (“IGS5DNA2”) of SEQ ID NO:5

5′-
ATGGGGAAGTCCGAAGGCCCAGTGGGGATGGTGGAGAGCGCCGGCCGTGCAGGGCAGAAG
CGCCCGGGGTTCCTGGAGGGGGGGCTGCTGCTGCTGCTGCTGCTGGTGACCGCTGCCCTG
GTGGCCTTGGGTGTCCTCTACGCCGACCGCAGAGGGATCCCAGAGGCCCAAGAGGTGAGC
GAGGTCTGCACCACCCCTGGCTGCGTGATAGCAGCTGCCAGGATCCTCCAGAACATGGAC
CCGACCACGGAACCGTGTGACGACTTCTACCAGTTTGCATGCGGAGGCTGGCTGCGGCGC
CACGTGATCCCTGAGACCAACTCAAGATACAGCATCTTTGACGTCCTCCGCGACGAGCTG
GAGGTCATCCTCAAAGCGGTGCTGGAGAATTCGACTGCCAAGGACCGGCCGGCTGTGGAG
AAGGCCAGGACGCTGTACCGCTCCTGCATGAACCAGAGTGTGATAGAGAAGCGAGGCTCT
CAGCCCCTGCTGGACATCTTGGAGGTGGTGGGAGGCTGGCCGGTGGCGATGGACAGGTGG
AACGAGACCGTAGGACTCGAGTGGGAGCTGGAGCGGCAGCTGGCGCTGATGAACTCACAG
TTCAACAGGCGCGTCCTCATCGACCTCTTCATCTGGAACGACGACCAGAACTCCAGCCGG
CACATCATCTACATAGACCAGCCCACCTTGGGCATGCCCTCCCGAGAGTACTACTTCAAC
GGCGGCAGCAACCGGAAGGTGCGGGAAGCCTACCTGCAGTTCATGGTGTCAGTGGCCACG
TTGCTGCGGGAGGATGCAAACCTGCCCAGGGACAGCTGCCTGGTGCAGGAGGACATGATG
CAGGTGCTGGAGCTGGAGACACAGCTGGCCAAGGCCACGGTACCCCAGGAGGAGAGACAC
GACGTCATCGCCTTGTACCACCGGATGGGACTGGAGGAGCTGCAAAGCCAGTTTGGCCTG
AAGGGATTTAACTGGACTCTGTTCATACAAACTGTGCTATCCTCTGTCAAAATCAAGCTG
CTGCCAGATGAGGAAGTGGTGGTCTATGGCATCCCCTACCTGCAGAACCTTGAAAACATC
ATCGACACCTACTCAGCCAGGACCATACAGAACTACCTGGTCTGGCGCCTGGTGCTGGAC
CGCATTGGTAGCCTAAGCCAGAGATTCAAGGACACACGAGTGAACTACCGCAAGGCGCTG
TTTGGCACAATGGTGGAGGAGGTGCGCTGGCGTGAATGTGTGGGCTACGTCAACAGCAAC
ATGGAGAACGCCGTGGGCTCCCTCTACGTCAGGGAGGCGTTCCCTGGAGACAGCAAGAGC
ATGGTCAGAGAACTCATTGACAAGGTGCGGACAGTGTTTGTGGAGACGCTGGACGAGCTG
GGCTGGATGGACGAGGAGTCCAAGAAGAAGGCGCAGGAGAAGGCCATGAGCATCCGGGAG
CAGATCGGGCACCCTGACTACATCCTGGAGGAGATGAACAGGCGCCTGGACGAGGAGTAC
TCCAATCTGAACTTCTCAGAGGACCTGTACTTTGAGAACAGTCTGCAGAACCTCAAGGTG
GGCGCCCAGCGGAGCCTCAGGAAGCTTCGGGAAAAGGTGGACCCAAATCTCTGGATCATC
GGGGCGGCGGTGGTCAATGCGTTCTACTCCCCAAACCGAAACCAGATTGTATTCCCTGCC
GGGATCCTCCAGCCCCCCTTCTTCAGCAAGGAGCAGCCACAGGCCTTGAACTTTGGAGGC
ATTGGGATGGTGATCGGGCACGAGATCACGCACGGCTTTGACGACAATGGCCGGAACTTC
GACAAGAATGGCAACATGATGGATTGGTGGAGTAACTTCTCCACCCAGCACTTCCGGGAG
CAGTCAGAGTGCATGATCTACCAGTACGGCAACTACTCCTGGGACCTGGCAGACGAACAG
AACGTGAACGGATTCAACACCCTTGGGGAAAACATTGCTGACAACGGAGGGGTGCGGCAA
GCCTATAAGGCCTACCTCAAGTGGATGGCAGAGGGTGGCAAGGACCAGCAGCTGCCCGGC
CTGGATCTCACCCATGAGCAGCTCTTCTTCATCAACTACGCCCAGGTGTGGTGCGGGTCC
TACCGGCCCGAGTTCGCCATCCAATCCATCAAGACAGACGTCCACAGTCCCCTGAAGTAC
AGGGTACTGGGGTCGCTGCAGAACCTGGCCGCCTTCGCAGACACGTTCCACTGTGCCCGG
GGCACCCCCATGCACCCCAAGGAGCGATGCCGCGTGTGGTAG - 3′

TABLE 6
IGS5-protein-2 (“IGS5PROT2”) of SEQ ID NO:6

MGKSEGPVGMVESAGRAGQKRPGFLEGGLLLLLLLVTAALVALGVLYADRRGIPEAQEVS
EVCTTPGCVIAAARILQNMDPTTEPCDDFYQFACGGWLRRHVIPETNSRYSIFDVLRDEL
EVILKAVLENSTAKDRPAVEKARTLYRSCMNQSVIEKRGSQPLLDILEVVGGWPVAMDRW
NETVGLEWELERQLALMNSQFNRRVLIDLFIWNDDQNSSRHIIYIDQPTLGMPSREYYFN
GGSNRKVREAYLQFMVSVATLLREDANLPRDSCLVQEDMMQVLELETQLAKATVPQEERH
DVIALYHRMGLEELQSQFGLKGFNWTLFIQTVLSSVKIKLLPDEEVVVYGIPYLQNLENI
IDTYSARTIQNYLVWRLVLDRIGSLSQRFKDTRVNYRKALFGTMVEEVRWRECVGYVNSN
MENAVGSLYVREAFPGDSKSMVRELIDKVRTVFVETLDELGWMDEESKKKAQEKAMSIRE
QIGHPDYILEEMNRRLDEEYSNLNFSEDLYFENSLQNLKVGAQRSLRKLREKVDPNLWII
GAAVVNAFYSPNRNQIVFPAGILQPPFFSKEQPQALNFGGIGMVIGHEITHGFDDNGRNF
DKNGNMMDWWSNFSTQHFREQSECMIYQYGNYSWDLADEQNVNGFNTLGENIADNGGVRQ
AYKAYLKWMAEGGKDQQLPGLDLTHEQLFFINYAQVWCGSYRPEFAIQSIKTDVHSPLKY
RVLGSLQNLAAFADTFHCARGTPMHPKERCRVW

All publications, including but not limited to patents and patent applications, cited in this specification are incorporated by reference herein as if each individual publication were specifically and individually indicated to be incorporated by reference herein and as though fully set forth.

The following examples are only intended to further illustrate the invention, in more detail, and therefore these examples are not deemed to restrict the scope of the invention in any way.

### EXAMPLE 1

### The Cloning of Cdna Encoding a Novel Member of the Neprilysin Nep/Ece Metalloprotease Family

### Example 1a

### Outline of Homology Cloning of cDNA Coding Sequence of IGS5

Metalloproteases of the M13 subfamily are involved in the metabolism of various neuronal and hormonal peptides. To date this subfamily comprises neprilysin (NEP), endothelin-converting enzyme-1 (ECE-1), ECE-2, Kell, Pex and XCE.

Inhibitors of NEP and ECE are being developed for therapeutical use for example in cardiology and gastroenterology. Since additional members of this family may be interesting drug targets, homology cloning was used to identify novel genes in the human genome.

Homology cloning of IGS5 was performed according to the general description supra and according to the experimental details further illustrated by the experimental section of the international patent application PCT/EP 00/11532 which is incorporated by reference herein. The procedure may be outlined as follows:

In the databank of expressed sequence tags (ESTs), sequences were detected which contained a small open reading frame that showed similarity to the C-terminal part of NEP/ECE-like metalloproteases. Based on these EST sequences and conserved peptide motifs of the NEP/ECE-like metalloproteases we used degenerate PCR to clone the complete cDNA sequence from human lung, heart and testis cDNA.

The cDNA sequence encoded a glycosylated protein, named IGS5 or alternatively human soluble endopeptidase (hSEP), displaying the characteristics of M13 family members. IGS5 showed high amino acid sequence identity to mouse SEP (78%), mouse, rat and human NEP (54%) and to human ECE-1 (39%). In analogy with the mouse SEP and SEPΔ splice variants we also detected two IGS5 splice forms which differed in a 78 bp alternative exon. These splice forms encoded proteins of 753 and 779 residues, respectively. The longer form contains a putative proteolytic cleavage site located adjacent to the transmembrane anchor. The two splice variants may therefore represent a membrane bound and a soluble form of the IGS5 protein.

Expression analysis using multiple tissue dot blot analysis and quantitative PCR revealed expression in a variety of human tissues, with the strongest signal observed in testis. IGS5 mRNA expression was also observed e.g. in prostate, small intestine, stomach, colon, kidney and brain. The two splice variants showed a distinct expression pattern.

The functional characterization confirmed that this enzyme is a genuine member of the neprylysin family, and possesses ECE activity.

### Example 1b

### Alignment of IGS5 with Protein Sequences of Members of the NEP/ECE Metalloprotease Family

For the IGS5 Sequence originally cloned (see example 1), homology searches of up to date protein databanks and translated DNA databanks were executed using the BLAST algorithm (Altschul S. F. et al. [1997], Nucleic Acids Res. 25:3389-3402). These searches showed that the originally obtained IGS5 protein was most similar (54-55% identities over ±700 aligned residues) to mouse, rat and human neutral endopeptidase (SW:NEP_MOUSE, accession no. Q61391; SW:NEP_RAT, accession no. P07861 and SW:NEP_HUMAN accession no. P08473). Thus, this alignment of the almost complete IGS5 protein sequence with the other members of the NEP/ECE family shows the relation of IGS5 to metalloproteases in general, and in particular to the NEP and/or ECE metalloprotease families. From this structural alignment it is concluded that the IGS5 has the functionality of metalloproteases, which in turn are of interest in the context of several dysfunctions, disorders or diseases in animals and humans.

### Example 1c

### Cloning of cDNA Fragments Containing the Full Length Coding Sequence of IGS5

In order to obtain additional IGS5 cDNA sequence another round of RT-PCR reactions was carried out on human lung RNA under the conditions described above using the IGS5 specific reverse primer. The resulting contig contained an open reading frame which started at an “ATG” initiation codon and encoded a protein which showed high similarity with the N-terminal sequence of the mouse SEP protein.

Assembly of the DNA sequences of all isolated clones showed the presence of two types of cDNA sequences, that differed by the presence or absence of the 78 bp segment, inititially identified within genomic clone IGS5/S1. These two sequences likely originate from alternatively spliced RNA molecules. The longest transcript contains an open reading frame of 2337 nucleotides (encoding a protein of 779 residues) whereas the shorter transcript contains an open reading frame of 2259 nucleotides (encoding a protein of 753 residues). The coding sequence and the protein sequence of the long form is referred to as IGS5DNA1 (shown in SEQ ID NO:3, 2340 bp including the stop codon tag) and IGS5PROT1 (SEQ ID NO:4) respectively, whereas the coding sequence and the protein sequence of the shorter form are referred to as IGS5DNA2 (shown in SEQ ID NO:5, 2262 bp including the stop codon tag) and IGS5PROT2 (SEQ ID NO:6) respectively. Downstream of the postulated methionine initiation codon within IGS5DNA1 and IGS5DNA2 an additional in-frame methionine codon is present at codon position 10. Although it is opted for the first methionine codon as being the initiaton codon some (or even exclusive) initiation of translation at codon position 10 cannot be excluded, since both methionines appear to be within an equally favorable “Kozak” initiation of translation context (Kozak M., Gene [1999]: 234: 187-208). Hydropathy analysis (Kyte J. et al., J. Mol. Biol. [1982] 157: 105-132; Klein P. et al., Biochim. Biophys. Acta [1985] 815: 468-476) of the IGS5PROT1 and IGS5PROT2 sequences showed the presence of a single transmembrane domain between residues 22 to 50. This indicates that IGS5PROT1 and IGS5PROT2 are type II integral membrane proteins and thus have a membrane topology similar to other members of the NEP/ECE protein family.

### Example 1c

### Alignment of IGS5 Protein Sequences of Example 1c with Protein Sequences of Members of the NEP/ECE Metalloprotease Family

For the IGS5 sequence cloned in example 1c, homology searches of up to date protein databanks and translated DNA databanks were executed using the BLAST algorithm (Altschul S. F. et al, Nucleic Acids Res. [1997] 25:3389-3402). These searches showed that IGS5PROT1 was most similar (76% identities over 778 aligned residues) to mouse SEP (GenBank accession no. AF157105) and also showed 54-55% identities over 696 aligned residues to mouse, rat and human neutral endopeptidases (SW:NEP_MOUSE, accession no. Q61391; SW:NEP_RAT, accession no. P07861; SW:NEP_HUMAN, accession no. P08473). Homology searches of IGS5PROT2 showed that this sequence was most similar (78% identities over 752 aligned residues) to mouse SEPΔ (GenBank accession no. AF157106). In analogy with the mouse SEP and SEPΔ proteins it is to be expected that IGS5PROT1 and IGS5PROT2 represent the soluble and membrane-bound forms of the IGS5 protein, respectively. This is corroborated by the presence of dibasic residues (KRK) encoded at the 3′ end of the alternatively spliced 78 bp exon.

Thus, this alignment of the complete IGS5 protein sequence with the other members of the NEP/ECE family shows the relation of IGS5 to NEP/ECE metalloproteases in general, and in particular to the SEP and NEP family members. From this structural alignment it is concluded that the IGS5 protein has the functionality of metalloproteases, which in turn are of interest in the context of several dysfunctions, disorders or diseases in animals and humans.

### EXAMPLE 2

### Expression and Purification of the Soluble His-Tagged Ectodomain of Human Igs5

The aim of the experiment was to produce soluble IGS5 protein using the baculoviral expression system. A recombinant baculovirus was constructed that expressed the His₆-tagged IGS5 ectodomain upon infection of the Sf9 cell-line. Soluble IGS5 protein was then purified from the culture supernatant in a two step procedure involving lentil-lectin and Zn-IMAC chromatography, as was done in the state of the art for His₆-ECE-1.

### Example 2a

### Experimental Procedures

Kinetic Expression Analysis.

519 cells (IGCL 83.0), exponentially growing in suspension in Spinner flasks at 27° C. in TC100 medium (JRH Biosciences cat n° 56941), supplemented with 10% inactivated Foetal Calf Serum (Gibco BRL cat n° 10 084 168), were collected by low speed centrifugation and seeded at 5.10⁵ cells/Fk (25 cm²) in serum-free TC100 medium. Candidate recombinant viral clones were added at a multiplicity of infection (MOI) of 3 pfu/cell and cell/virus cultures were subsequently incubated at 27° C. Cells and conditioned medium (CM) were harvested at 24, 48 and 72 h post infection by 2 consecutive low speed centrifugations. Samples were analyzed by SDS PAGE gel electrophoresis and Western blotting.

Deglycosylation Study.

Samples were supplemented with SDS to a final concentration of 1% and incubated at 95° C. for 5 min. After addition of 1 volume of the 2× incubation buffer (250 mM phosphate buffer, 50 mM EDTA, 5% N-octylglycoside, 1% 2-mercaptoethanol) and an additional 5 min incubation time at 95° C., the sample was cooled to 37° C. 1 U of N-glycosidase F (Boehringer Mannheim, cat n° 1 365 177) was added and after overnight incubation at 37° C., the sample was reduced with 100 mM DTT (final concentration).

Preparative Production.

519 cells (IGCL 83-2) exponentially growing in suspension in spinner flasks at 27° C. in TC100 medium (JRH Biosciences, cat n° 56941) supplemented with 10% inactivated Foetal Calf Serum (Gibco BRL, cat n° 10 084 168) were collected by low speed centrifugation and resuspended at a density of 2.10⁶ cells/ml in TC100 medium, supplemented with 0.013 TIU aprotinin/ml (Pentex). Recombinant virus IGBV73 was added to the cells at a multiplicity of infection (MOI) of 2.25 pfu/cell (in stead of MOI 3 due to the low titer of the primary virus bank). The cell/virus suspension was subsequently incubated at 27° C. in glass roller bottles (3×500 ml/1260 cm²) for 72 h. The CM (1.5 l) was then cleared from cells and cell debris by two consecutive low speed centrifugations. Aliquots were taken for quality control by Western blot analysis and for the determination of endotoxin levels.

### Example 2b

### Results

Kinetics of Expression.

The kinetics of expression of three candidate recombinant viral clones were studied via Western blot analysis. Western blot revealed a clear band at approximately 81 kDa in the CM of all candidate clones, corresponding to the theoretical Mr of the mature protein (81.2 kDa). For cell lysates, the SDS gel was overloaded so no conclusions could be drawn. Expression levels of all 3 clones peaked at 48 to 72 h post-infection. Clone 2 was selected for further amplification and was deposited as IGBV73. Optimal harvest time was set at 72 h post infection.

Deglycosylation Study.

The soluble IGS5 protein sequence contains 8 potential N-glycosylation sites. Since the purification protocol involves binding of the sugar residues on a lentil-lectin column samples of CM and cell lysates, harvested at 72 h post infection were used for a deglycosylation study with N-glycosidase F, to check whether the recombinant soluble His₆IGS5 protein is indeed expressed as a glycosylated protein.

Western blot analysis of N-glycosidase F treated CM samples and non-treated controls show a shift in Mr when samples are deglycosylated, demonstrating that the soluble human His-tagged IGS5 is expressed as a glycosylated protein. In the non treated cell lysates, 3 protein bands of approximately 80 to 82 kDa can be observed. Upon N-glycosidase F treatment, 1 band of approximately 80 kDa remains visible, corresponding to the lowest MW band of the non treated samples.

Preparative Production.

1.5 liter of CM was harvested from IGBV73 infected Sf9 insect cells 72 h post infection.

Endotoxin content was determined to be 0.0847 EU/ml CM. Western blot analysis revealed a clear band at approximately 81 kDa in the CM, corresponding to the MW of the mature soluble His-tagged IGS5. When compared to cell lysate samples, which showed 3 protein bands, the CM protein band corresponds to the weaker middle Mr band, present in the cells.

### EXAMPLE 3

### Purification of Igs5

### Example 3a

### Experimental Procedures

Sample Pretreatment.

1 tablet of EDTA free complete (EFC; Roche Biochemicals, cat n° 1873580) was added to 300 ml cleared Baculo CM. HEPES, glycerol and Tween 20 were added to a final concentration of resp. 20 mM, 5% (v/v) and 0.005% (w/v). The pH of the CM was adjusted to 7.4 and the sample was filtrated Durapore Membrane Filters 0.2 μGV). All purification steps were performed at 4° C.

Lentil Lectin Chromatography.

The baculo sample was loaded overnight at 0.3 ml/min on a 5 ml Lentil Lectin Sepharose resin in a C10/10 column (Pharmacia), which had been equilibrated in buffer A (20 mM Hepes, 150 mM NaCl, 5% glycerol, 0.005% Tween 20) supplemented with 1 tablet EFC/500 ml. The column was washed with equilibration buffer until the absorbance at 280 nm reached baseline level and the bound proteins were eluted by applying buffer A containing 0.5 M alpha-methylpyrrannoside. The column was regenerated by applying 100 mM acetate, 500 mM NaCl, pH 5.0. The elution and regeneration liquids were collected manually and the pools were analyzed by SDS-PAGE on 12.5% Phast gels (Pharmacia) and silver staining. Prestained markers (Gibco) were included as relative molecular weight (Mr) standard.

Immobilized Metal Affinity Chromatography (IMAC) and Dialysis.

1 ml Chelating HiTrap (Pharmacia) was loaded with zinc ions as described by the manufacturer and equilibrated with buffer B (20 mM Hepes, 100 mM NaCl, 5% glycerol, 0.005% (w/v) Tween 20, pH 7.2). Lentil elution pools 1 and 2 were loaded separately at 0.5 ml/min on the HiTrap column (IMAC run A and IMAC run B). A blank run was included to compare the chromatographic absorbance profile. The column was washed with buffer B till baseline level and bound proteins were eluted by applying an imidazole step gradient (20, 50, 100 and 200 mM) in buffer B. Fractions were collected manually. The IMAC column was regenerated by applying 20 mM Hepes, 50 mM EDTA, 500 mM NaCl, pH 7.2. Elution and regeneration pools were analyzed by SDS-PAGE (12.5% Phast gels, Pharmacia) and silver staining. The 200 mM imidazole pool was transferred to a slide a-lyzer-cassette (MWCO 10.000, Pierce) and dialyzed overnight against buffer B (130 fold excess, no buffer refreshment).

Protein Quantification.

The amount of soluble IGSS in the dialyzed pool was determined with the micro-BCA method (Pierce). BSA was included as reference.

Protein Characterization.

The dialyzed baculo IGSS was biochemically characterized by (1) SDS-PAGE under reducing and non reducing conditions and (2) Western blot with an anti His-tag mAb (21E1B4, IG) followed by incubation with alkaline phosphatase labeled rabbit anti-mouse Ig (Dako) and detection with NBT/BCIP staining. The glycosilation status of the soluble IGS5 was verified by PGNase F treatment (Biorad).

### Example 3b

### Results

The Lentil elution profile with 0.5 M alfa-methylpyrrannoside resulted in a tailing peak. The flow through, washes and the elution pools were analyzed by SDS-PAGE and silver staining. The major amount of proteins were retrieved in the flow through and an IGS5-candidate band with a Mr of about 85.000 was observed in elution pools 1 to 3. Western blot analysis of the lentil chromatography with the anti-His tag mAb showed that the soluble hlGS5 protein (Mr˜85.000) is quantitatively bound to the Lentil Lectin resin and that the His-tagged protein is recovered over the whole elution peak, but mainly in pools 1 and 2. The Lentil Lectin elution pools 1 and 2 were further processed on the zinc-IMAC column (runs A and B). The bound proteins were eluted by an imidazole step gradient. SDS-PAGE analysis and silver staining showed that the bulk of contaminating proteins were eluted by applying the 20 mM and 50 mM imidazole step. The hlGS5 protein was retrieved in the 100 mM and 200 mM imidazole elution steps. The 100 mM imidazole elution, which contains maximum 10% of the hlGS5 in the eluate, is still contaminated with a protein with a Mr of >115.000. The IGS5 band in the 100 mM is also a doublet band. It remains to be verified whether the faint upper band, which represents less than 10% of the doublet, is a residual baculo contaminant or an IGS5 isoform or whether the lower (intense) band is a carboxyterminal degradation product. The 85 kDa band in the 200 mM imidazole pool is a single band on the SDS-PAGE, which reacts with the anti his-tag mAb.

Silver staining did not reveal any difference in purity between the hlGS5 material obtained from IMAC run A and run B. This indicates that pool 1 and pool 2 of the Lectin eluate can be pooled in the future and be processed simultaneously in a single run on the zinc-IMAC column.

An identical band pattern was observed on Coomasssie stained SDS-PAGE gels run under reducing and non reducing conditions, indicating that the purified hlGS5 does not contain disulfide based oligomers. Treatment with PGNase F reduced the Mr on SDS-PAGE with about 5 kDa. This minimal shift of the baculo expressed hlGS5 after treatment with endoglycosidase differs strongly from the migration shift which was observed for CHO expressed mouse SEP (Ikeda et al., JBC, 274, 32469, 1999).

Starting from 300 ml of baculo CM, 340 μg of over 95% pure His-tagged hlGS5 ectodomain was obtained by the 2 step purification procedure (i.e. a yield of about 1 mg/l). The purified product was then used in the enzyme inhibition assays as indicated in the following examples.

### EXAMPLE 4

### Igs5 Enzyme Inhibition Assay

The enzymatic activity of IGS5 polypeptides of the invention was tested with regard to the metabolism of biologically active peptides. In particular it was tested whether these IGS5 polypeptides may act on a variety of vasoactive peptides known in the state of the art e.g. such like atrial natriuretic peptide (ANP), bradykinin, big-endothelin (big-ET-1), endothelin (ET-1), substance P and angiotensin-1. In the context of the present invention in particular it was tested whether the IGS5 ectodomain, which is a novel human metalloprotease, hydrolyzes said vasoactive peptides. For comparison the assay was also performed for a known member of the metalloprotease family which was described earlier as soluble secreted endopeptidase (SEP) by Emoto et al. (J. Biol. Chem., Vol. 274 (1999): pp. 32469-32477). Furthermore, it was tested whether the activity of IGS5 to convert a big-ET-1 analog (the so-called 17 aa big-ET-1) may be inhibited by reference compounds that are used to determine the inhibition properties with regard to enzymes having ECE and/or NEP-characteristics. Compounds used to test the inhibition of IGS5-activity on the big-ET-1 analog were the compound phosphoramidon which inhibits endopeptidases with NEP-characteristics, the compound thiorphan which selectively inhibits NEP, and the compound CGS-35066 which is a dual NEP/ECE inhibitor.

### Example 4a

### Materials

Enzyme: IGS-5 (sol hu)(his)₆; or: His6-tagged IGS5 ectodomain;

stock solution: 53 μg/ml in 20 mM HEPES pH 7.2, 5% glycerol, 0.005% Tween20, 100 mM NaCl, purity >99%; storage at 4° C.

working solution: stock solution diluted with assay buffer to 10 μg/ml.

Substrate: Mca-Asp-Ile-Ala-Trp-Phe-Dpa-Thr-Pro-Glu-His-Val-Val-Pro-Tyr-Gly-Leu-Gly-COOH;

Fluorescence-quenched big-ET-1 analog;

Mca=7-Methoxycoumarin-4-yl;

Dpa=3-[2,4-Dinitrophenyl]-L-2,3-diaminopropionyl;

stock solution: 100 μM in assay buffer; storage at −20° C. (commercially available from supplier: Polypeptide Laboratories, Wolfenbüttel, Germany)

Assay buffer: 100 mM Tris pH 7.0, 250 mM NaCl.

All test compounds were dissolved in DMSO at 10 mM and were further diluted with assay buffer.

### Example 4b

### Assay Procedure

A quantity of 70 μl of the assay buffer, of 10 μl enzyme working solution and of 10 μl test compound solution were mixed in an Eppendorf vial and preincubated at 37° C. for 15 minutes. Then, 10 μl substrate stock solution was added and the reaction mixture was incubated at 37° C. for 60 minutes to allow for enzymatic hydrolysis. Subsequently the enzymatic reaction was terminated by heating at 95° C. for 5 minutes. After centrifugation (Heraeus Biofuge B, 3 min) the supernatant was subjected to HPLC analysis.

### Example 4c

### HPLC Procedure

In order to separate the remaining substrate from the cleavage products reversed phase HPLC technique was used with a CC 125/4 Nucleosil 300/5 C₁₈ RP column and a CC 8/4 Nucleosil 100/5 C18 precolumn (commercially available from Macherey-Nagel, Düren, Germany). Thus, 60 μl of the reaction samples obtained in Example 7b were injected into the HPLC, and the column was eluted at a flow rate of 1 ml/min by applying the following gradient and solutions:

Solution A: 100% H₂O+0.5 M H₃PO₄, pH=2.0

Solution B: 100% acetonitrile+0.5M H₃PO₄

0-2 min 20% B

2-6 min 20-60% B

6-8 min 60% B

8-10 min 60-90% B

10-13 min 90% B

13-15 min 90-100% B

Peptides were detected by absorbance at 214 nm and by fluorescence with an excitation wavelength of 328 nm and an emission wavelength of 393 nm.

### Example 4d

### Calculations

The increasing fluorescence signal of the HPLC-peak of the peptide with the unquenched Mca-fluorophor after hydrolysis was taken as the basis for any calculation.

This signal was compared for the samples with and without inhibitor and % inhibition was calculated on basis of the respective peak areas.

% inhib=100*(1−A_{inhib}/A_{control})

All samples were run in duplicate and mean values were used.

A standard inhibitor (10 nM and 100 nM Phosphoramidon) and a solvent control (0.1%) was added to each assay run.

### Example 4e

### Results

With regard to the IGS5 polypeptides of the present invention the results of Example 4 show that these IGS5 metalloprotease polypeptides hydrolyze in vitro a variety of vasoactive peptides known in the state of the art. The results of the hydrolysis assay in comparison to the activity of SEP are shown in Table 7. From these results it is concluded that IGS5 may be particularly involved in the metabolism of said biologically vasoactive peptides.

TABLE 7
Hydrolysis of vasoactive peptides by IGS5 polypeptides in
comparison to SEP (soluble secreted endopeptidase).

| | % Hydrolysis | % Hydrolysis |
|---|---|---|
| | by IGS5 Polypeptide | by SEP (Emoto et al.) |
| | Conditions: | Conditions: |
| | 100 μg IGS5 polypeptide; | 10 μg SEP; |
| Vasoactive | 0.5 μM substrate; | 0.5 μM substrate; |
| Peptide | 2 h, 37° C. | 12 h, 37° C. |
| ANP | 5 (80*) | >95 |
| Bradykinin | 100 (62**) | >95 |
| ET-1 | <30 | 92 |
| Substance P | 100 | >95 |
| Angiotensin 1 | 15*** | >95 |
| 17 aa big-ET-1**** | 41 | n.d. |

*500 μg IGS5 polypeptide
**10 μg IGS5 polypeptide
***degradation of angiotensin I does not result in formation of angiotensin II
****17 aa big-ET-1 was used as an analog of the natural big-ET-1; hydrolyzing activity of IGS5 was also detected using the natural big-ET-1, but could not be quantified due to difficulties with the HPLC-detection

### EXAMPLE 5

### Nep Enzyme Inhibition Assay

Neutral endopeptidase (E.C. 3.4.24.11) was prepared from pig kidney cortex according to the method of Gee et al. (Biochem J 1985 May 15;228(1):119-26) and purified as reported by Relton et al. (Biochem J 1983 Dec. 1;215(3):519-23). For the enzyme inhibition assay 10 ng of the purified enzyme, 20 μM substrate (methionin-enkephalin) and various inhibitor concentrations were used. The assay buffer was 50 mM Tris-(hydroxymethyl)-aminomethan/HCl pH 7.4, the total assay volume was 100 μl. After a preincubation period of 5 min of enzyme and inhibitor, the substrate was added and subsequently a second incubation phase for enzyme induced substrate hydrolysis at 37° C. for 30 min was initiated. The enzymatic reaction was stopped by heating at 95° C. for 5 min. After centrifugation the supernatant was subjected to HPLC. The products of enzymatic substrate hydrolysis were separated from the native substrate by HPLC technique and the inhibitor potency calculated by comparing the peak areas of the products with the peak area of native substrate for both, the samples with and without inhibitor (control). Blanks without enzyme, controls without inhibitor, samples with inhibitor solvent instead of the inhibitor and samples with a standard inhibitor were added to each assay run.

Supplier of Materials:

NEP: Dr. Philippe Crine, Univ. of Montreal, Canada

Methionin-enkephalin: Sigma, Deisenhofen, Germany

### EXAMPLE 6

### Ece Enzyme Inhibition Assay

Recombinant human COOH-terminal His6-tagged endothelin converting enzyme-1 was expressed in Sf9-cells. Purification was performed by affinity chromatography. The enzyme inhibition assay comprised enzyme (2.8 μg), 5 μg substrate (moderately modified 17 amino acid truncated big endothelin-1), inhibitor at various final concentrations and 100 mM Tris-buffer (Tris-hydroxymethl-aminomethan/HCl, pH 7.0+150 mM NaCl) in a final volume of 100 μl. Pre-incubation of enzyme with inhibitor for 15 min at 37° C. was performed before substrate addition and incubation (60 min at 37° C.) for enzymatic hydrolysis. The enzymatic reaction was stopped by heating at 95° C. for 5 min. After centrifugation the supernatant was subjected to HPLC for separation of enzymatic hydrolysis products from undegraded substrate. % inhibition was calculated on the basis of peak areas for products and uncleaved substrate for the inhibited reaction in comparison to the control (without inhibitor). Blanks without enzyme, controls without inhibitor, samples with inhibitor solvent instead of the inhibitor and samples with a standard inhibitor were added to each assay run.

Supplier of Materials:

ECE: Innogenetics, Ghent, Belgium

ECE substrate: Polypeptide, Wolfenbüttel, Germany

### EXAMPLE 7

### Biochemical Profile of Nep/Igs5-Inhibitory Compounds in Comparison to Reference Compounds

In order to characterize and evaluate the pharmacological enzymatic properties of IGS5 for the purpose of the present invention a human IGS5 protein was generated by using an insect cell line as the expression system as described in the examples supra, and a variety of potential substrates of the IGS5 protein were tested. According to the results of example 4 IGS5 was found to efficiently cleave big-ET-1, ANP, and bradykinin, thus confirming that this novel protein is a genuine metalloprotease with a broad substrate specificity, which is a common feature of metalloproteases and which feature has been reported for NEP, ECE-1 and also ACE. It should also be noted that according to the findings of the present invention the proteolysis of big-ET-1 by IGS5 surprisingly results in the correct formation of ET-1, e.g. big-ET-1 is correctly cleaved between amino acids Trp21 and Val22.

Furthermore, the potency of metalloprotease inhibitor compounds and reference compounds to suppress the conversion of big-ET to ET-1 was examined as described in this example 7, using a labeled fluorescent big-ET-1 analog. The procedures applied in the enzyme assays are described above in the examples 4 with regard to IGS5, in example 5 with regard to NEP, and in example 6 with regard to ECE.

The results of the enzyme inhibition tests are summarized in Table 8. It is of interest that phosphoramidon that is known to inhibit the conversion of big-ET to ET-1 in vivo, also inhibits IGS5 with high potency in the biochemical assay used in the present invention, and surprisingly that the inhibition of IGS5 by phosphoramidon is actually considerably higher than ECE-1. In contrast, the selective NEP inhibitor thiorphan as well as the selective ECE-1 inhibitor SM-19712 (4-chloro-N-[[(4-cyano-3-methyl-1-phenyl-1H-pyrazol-5-yl)amino]carbonyl]benzenesulfonamide, monosodium salt; Umekawa K, Hasegawa H, Tsutsumi Y, Sato K, Matsumura Y, Ohashi N., J Pharmacol 2000 September; 84(1):7-15; Discovery Research Laboratories I, Research Center, Sumitomo Pharmaceuticals Co, Ltd, Osaka, Japan) do not affect the activity of IGS5 (Table 8).

TABLE 8
Biochemical profile of NEP/IGS5-inhibitory compounds
and reference compounds

| | IGS5 | NEP | ECE-1 |
|---|---|---|---|
| Compound | IC50/nM | IC50/nM | IC50/nM |
| Phosphoramidon | 18 | 2.0 | 300 |
| CGS-35066 | 1300 | 42 | 5 |
| Thiorphan | >1000 | 2.4 | >10000 |
| SM-19712 | >10000 | >1000 | 11.7 |
| compound Ia-2 | 1.2 | 2.9 | 1000 |
| active drug | | | |
| compound Ia-2 | 2.9 | 1.7 | 3279 |
| active drug | | | |
| compound Ia-2 | 2.8 | 4 | 374 |
| active drug | | | |

The results of the present invention are very surprising, in particular in view of the following facts.

Since endothelin converting enzyme-1 (ECE-1) was cloned in 1994, this enzyme has become generally accepted as the endopeptidase responsible for the physiological conversion of big-ET-1 to ET-1. However, despite the fact that ECE-1 has the ability to cleave big-ET-1, more recent reports raise doubts as to whether ECE-1 is the only physiologically relevant endothelin converting enzyme, or at least argue that additional enzymes must be involved in the production of ET-1. The IGS5 protein was discovered to be a metalloprotease with high similarity to NEP (54% identity) and a somewhat lower similarity to ECE-1 (39% identity). To characterize the enzymatic properties of this novel metalloprotease by the experiments under the example 4 it was looked for potential substrates and found that substance P, bradykinin, big-ET-1 and less efficiently ANP, angiotensin I and ET-1 are cleaved by IGS5. The enzymatic activity of IGS5 has an optimum at neutral pH (7.0-7.5). It should be noted that the proteolysis of big-ET-1 by IGS5 results in correct formation of ET-1.

By the experiments under the present example 7 the potency of metalloprotease inhibitors to suppress the conversion of big-ET to ET-1 by IGS5 was examined, using a labeled fluorescent big-ET-1 analog as a substrate. It is of interest that phosphoramidon that is known to inhibit the conversion of big-ET to ET-1 in vivo, also inhibits IGS5 with high potency (IC50=18 nM) in our biochemical assay (considerably higher than ECE-1). In contrast, the selective NEP inhibitor thiorphan as well as a selective ECE-1 inhibitor SM-19712 from Sumitomo do not affect the activity of IGS5.

Thus compounds with formula I, in particular with formula Ia or Ib, respectively, surprisingly showed combined or concurrent NEP/IGS5-inhibitory activity. As representative examples of compounds of the formula Ia compound Ia-2 and of the formula Ib compound Ib-8 were investigated in the experiments of the present example 7. Both compounds are very potent inhibitors (IC50=1.2 and 2.9 nM) of the newly identified IGS5 metalloprotease.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A method of treating or inhibiting a disease state in a mammal which can be alleviated by concurrent inhibition of neutral endopeptidase and the metalloprotease IGS5, wherein said metalloprotease IGS5 is a polypeptide comprising an amino acid sequence which has at least 70% identity to an amino acid sequence selected from the group consisting of SEQ ID NO:2, SEQ ID NO:4 and SEQ ID NO:6, said method comprising administering to said mammal an effective amount of a compound, or pharmaceutically acceptable salt thereof, having inhibitory activity
a) on neutral endopeptidase and
b) on said metalloprotease IGS5.

2. The method of claim 1, wherein said compound is present as a solvate or as a biolabile ester.

3. The method of claim 1, wherein said disease state is characterized by elevated big-ET-1 levels and wherein said disease state can be alleviated or inhibited by administering an effective amount of a compound having combined inhibitory activity on neutral endopeptidase and on IGS5.

4. The method of claim 1, wherein said disease state is characterized by upregulation of ET-1 and wherein said disease state can be alleviated or inhibited by administering an effective amount of a compound having combined inhibitory activity on neutral endopeptidase and on IGS5.

5. The method of claim 1, wherein said disease state includes at least one condition selected from the group consisting of hypertension, heart failure, angina pectoris, arrhythmias, myocardial infarction, cardiac hypertrophy, cerebral ischemia, peripheral vascular disease, subarachnoidal hemorrhage, chronic obstructive pulmonary disease, asthma, renal disease, atherosclerosis, and pain in colorectal cancer or prostate cancer.

6. The method of claim 5, wherein said disease state is renal hypertension or pulmonary hypertension.

7. The method of claim 1, wherein said metalloprotease is a polypeptide comprising an amino acid sequence which has at least 95% identity to an amino acid sequence selected from the group consisting of SEQ ID NO:2, SEQ ID NO:4 and SEQ ID NO:6.

8. The method of claim 1, wherein said metalloprotease is a polypeptide comprising an amino acid sequence which is identical to an amino acid sequence selected from the group consisting of SEQ ID NO:2, SEQ ID NO:4 and SEQ ID NO:6.

9. A method of treating or inhibiting a disease state in a mammal which can be alleviated by inhibiting neutral endopeptidase and the metalloprotease IGS5, wherein said metalloprotease IGS5 is a polypeptide comprising an amino acid sequence which has at least 70% identity to an amino acid sequence selected from the group consisting of SEQ ID NO:2, SEQ ID NO:4 and SEQ ID NO:6, said method comprising administering to said mammal an effective amount of a compound corresponding to formula I, 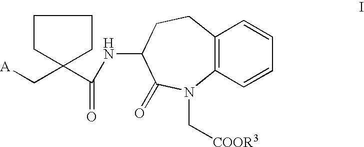
wherein
A corresponds to formula II or III 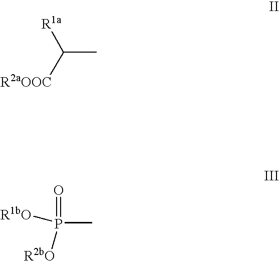
wherein in formula II
R^{1a} is a phenyl-lower-alkyl group or a naphthyl-lower-alkyl group,
R^{2a} is hydrogen or a group forming a biolabile ester; and
wherein in formula III
R^{1b} is hydrogen or a group forming a biolabile phosphonic acid ester,
R^{2b} is hydrogen or a group forming a biolabile phosphonic acid ester; and
R³ is hydrogen or a group forming a biolabile ester;
or a pharmaceutically acceptable salt thereof.

10. The method of claim 9, wherein R^{1a} is a phenyl-lower-alkyl group which is substituted in the phenyl ring by lower alkyl, lower alkoxy or halogen.

11. The method of claim 9, wherein R³ is a group forming a biolabile carboxylic acid ester.

12. The method of claim 9, wherein said compound is in the form of a solvate or of a biolabile ester.

13. The method of claim 9, said method comprising administering to said mammal an effective amount of a compound corresponding to formula Ia, 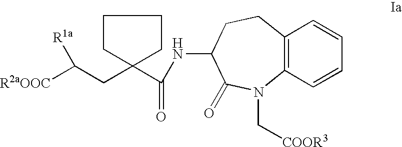
or a pharmaceutically acceptable salt thereof.

14. The method of claim 13, wherein R^{1a} is a phenyl-lower-alkyl group which is substituted in the phenyl ring by lower alkyl, lower alkoxy or halogen.

15. The method of claim 13, wherein said compound is in the form of a solvate.

16. The method of claim 9, said method comprising administering to said mammal an effective amount of a compound corresponding to formula Ib, 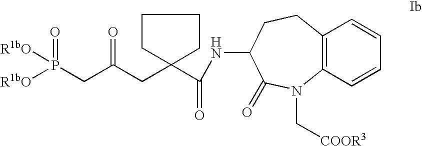
or a pharmaceutically acceptable salt thereof.

17. The method of claim 16, wherein R³ is a group forming a biolabile carboxylic acid ester.

18. The method of claim 16, wherein said compound is in the form of a solvate.

19. The method of claim 9, wherein said disease state is characterized by elevated big-ET-1 levels.

20. The method of claim 9, wherein said disease state is characterized by upregulation of ET-1.

21. The method of claim 9, wherein said disease state includes at least one condition selected from the group consisting of hypertension, heart failure, angina pectoris, arrhythmias, myocardial infarction, cardiac hypertrophy, cerebral ischemia, peripheral vascular disease, subarachnoidal hemorrhage, chronic obstructive pulmonary disease, asthma, renal disease, atherosclerosis, and pain in colorectal cancer or prostate cancer.

22. The method of claim 21, wherein said disease state is renal hypertension or pulmonary hypertension.

23. The method of claim 19, wherein said disease state includes at least one condition selected from the group consisting of hypertension, heart failure, angina pectoris, arrhythmias, myocardial infarction, cardiac hypertrophy, cerebral ischemia, peripheral vascular disease, subarachnoidal hemorrhage, chronic obstructive pulmonary disease, asthma, renal disease, atherosclerosis, and pain in colorectal cancer or prostate cancer.

24. The method of claim 20, wherein said disease state includes at least one condition selected from the group consisting of hypertension, heart failure, angina pectoris, arrhythmias, myocardial infarction, cardiac hypertrophy, cerebral ischemia, peripheral vascular disease, subarachnoidal hemorrhage, chronic obstructive pulmonary disease, asthma, renal disease, atherosclerosis, and pain in colorectal cancer or prostate cancer.

25. The method of claim 9, wherein said metalloprotease is a polypeptide comprising an amino acid sequence which has at least a 95% identity to an amino acid sequence selected from the group consisting of SEQ ID NO:2, SEQ ID NO:4 and SEQ ID NO:6.

26. The method of claim 9, wherein said metalloprotease is a polypeptide comprising an amino acid sequence which is identitical to an amino acid sequence selected from the group consisting of SEQ ID NO:2, SEQ ID NO:4 and SEQ ID NO:6.

27. The method of claim 1, further comprising administering to said mammal an additional metalloprotease inhibitor.

28. The method of claim 27, wherein said additional metalloprotease inhibitor is selected from the group consisting of angiotensin converting enzyme inhibitors, selective endothelin converting enzyme inhibitors, selective neutral endopeptidase inhibitors, dual neutral endopeptidase/endothelin converting enzyme inhibitors, and mixed inhibitors of these metalloproteases.

29. The method of claim 27, wherein said compound having inhibitory activity and said additional metalloprotease inhibitor are co-effective.

30. The method of claim 27, wherein said compound having inhibitory activity and said additional metalloprotease inhibitor are synergistically effective.

31. The method of claim 9, further comprising administering to said mammal an additional metalloprotease inhibitor.

32. The method of claim 31, wherein said additional metalloprotease inhibitor is selected from the group consisting of angiotensin converting enzyme inhibitors, selective endothelin converting enzyme inhibitors, selective neutral endopeptidase inhibitors, dual neutral endopeptidase/endothelin converting enzyme inhibitors, and mixed inhibitors of these metalloproteases.

33. The method of claim 31, wherein the compound corresponding to formula I and the additional metalloprotease inhibitor are co-effective.

34. The method of claim 31, wherein the compound corresponding to formula I and the additional metalloprotease inhibitor are synergistically effective.

35. The method of claim 13, further comprising administering to said mammal an additional metalloprotease inhibitor.

36. The method of claim 35, wherein said additional metalloprotease inhibitor is selected from the group consisting of angiotensin converting enzyme inhibitors, selective endothelin converting enzyme inhibitors, selective neutral endopeptidase inhibitors, dual neutral endopeptidase/endothelin converting enzyme inhibitors, and mixed inhibitors of these metalloproteases.

37. The method of claim 35, wherein the compound corresponding to formula Ia and the additional metalloprotease inhibitor are co-effective.

38. The method of claim 35, wherein the compound corresponding to formula Ia and the additional metalloprotease inhibitor are synergistically effective.

39. The method of claim 16, further comprising administering to said mammal an additional metalloprotease inhibitor.

40. The method of claim 39, wherein said additional compound is a metalloprotease inhibitor selected from the group consisting of angiotensin converting enzyme inhibitors, selective endothelin converting enzyme inhibitors, selective neutral endopeptidase inhibitors, dual neutral endopeptidase/endothelin converting enzyme inhibitors, and mixed inhibitors of these metalloproteases.

41. The method of claim 39, wherein the compound corresponding to formula Ib and the additional compound are co-effective.

42. The method of claim 39, wherein the compound corresponding to formula Ib and the additional metalloprotease inhibitor are synergistically effective.

43. A pharmaceutical composition for inhibiting at least one metalloprotease, comprising:
a first compound having inhibitory activity for neutral endopeptidase and inhibitory activity for the metalloprotease IGS5, wherein said metalloprotease IGS5 is a polypeptide comprising an amino acid sequence which has at least 70% identity to an amino acid sequence selected from the group consisting of SEQ ID NO:2, SEQ ID NO:4 and SEQ ID NO:6, or a pharmaceutically acceptable salt thereof;
at least one additional metalloprotease inhibitor, and
a pharmaceutically acceptable carrier.

44. The pharmaceutical composition of claim 43, wherein said additional metalloprotease inhibitor is selected from the group consisting of angiotensin converting enzyme inhibitors, selective endothelin converting enzyme inhibitors, selective neutral endopeptidase inhibitors, dual neutral endopeptidase/endothelin converting enzyme inhibitors, and mixed inhibitors of these metalloproteases.

45. The pharmaceutical composition of claim 43, wherein said first compound and said at least one additional metalloprotease inhibitor are co-effective.

46. The pharmaceutical composition of claim 43, wherein said first compound and said at least one additional metalloprotease inhibitor are synergistically effective.

47. The pharmaceutical composition of claim 43, wherein said first compound is in the form of a solvate or of a biolabile ester.

48. The pharmaceutical composition of claim 43, wherein said first compound corresponds to the structure of formula I, 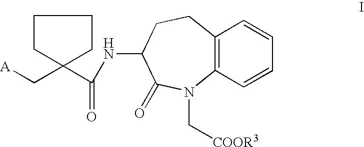
wherein
A corresponds to formula II or III 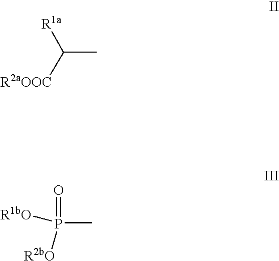
wherein in formula II
R^{1a} is a phenyl-lower-alkyl group or a naphthyl-lower-alkyl group,
R^{2a} is hydrogen or a group forming a biolabile ester; and
wherein in formula III
R^{1b} is hydrogen or a group forming a biolabile phosphonic acid ester,
R^{2b} is hydrogen or a group forming a biolabile phosphonic acid ester; and
R³ is hydrogen or a group forming a biolabile ester;
or a pharmaceutically acceptable salt thereof.

49. The pharmaceutical composition of claim 48, wherein said first compound corresponds to the structure of formula Ia, 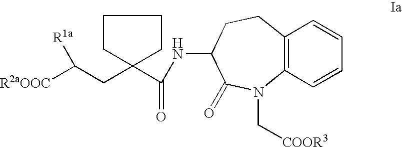
or a pharmaceutically acceptable salt thereof.

50. The pharmaceutical composition of claim 48, wherein said first compound corresponds to the structure of formula Ib, 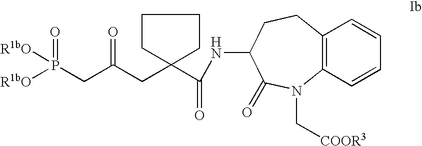
or a pharmaceutically acceptable salt thereof.
